(19) United States
(12) Patent Application Publication
(10) Pub. No.: US 2016/0228483 A1
(43) Pub. Date: Aug. 11, 2016

(54) METHOD FOR EXTRACTION AND CONCENTRATION OF ALKALOIDS USING DIMETHYL ETHER

(71) Applicant: NORAMCO, INC., Athens, GA (US)

(72) Inventors: Douglas Phillip Cox, Eagleville, PA (US); Timothy Samuel Bailey, Tasmania (AU); Val Krukonis, Lexington, MA (US); Robert Rezaie, Tasmania (AU); Hans Schonemann, Newburyport, MA (US); Brian Jeffrey Waibel, Kennett Square, PA (US)

(21) Appl. No.: 14/860,608

(22) Filed: Sep. 21, 2015

Related U.S. Application Data
(60) Provisional application No. 62/057,321, filed on Sep. 30, 2014.

(51) Int. Cl. A61K 36/66; A61K 31/485

(52) U.S. Cl. CPC A61K 36/66; A61K 31/485; A61K 2236/10; A61K 2236/53; A61K 2236/55; A61K 2236/51; A61K 2236/37

(57) ABSTRACT

The present invention is directed to a method for the extraction and/or enrichment of alkaloids from a mixture containing such compounds. More particularly, the present invention is directed to methods for extracting one or more alkaloids using dimethyl ether.

### CROSS REFERENCE TO RELATED APPLICATIONS

This application claims the benefit of U.S. Provisional Application No. 62/057,321, filed on Sep. 30, 2014, which is incorporated by reference herein in its entirety.

### FIELD OF THE INVENTION

The present invention is directed to a method for the extraction and/or enrichment of morphinan alkaloids or benzylisoquinoline alkaloids from a mixture containing such compounds. More particularly, the present invention is directed to methods for extracting one or more morphinan alkaloids and/or one or more benzylisoquinoline alkaloids from botanical and biological substrates, such as the poppy straw resulting from the end of the growth cycle of the Papaver somniferum poppy, or mutants thereof, the roots and straw from the Papaver bracteatum poppy, opium and tissue cultures of plant material enriched in secondary metabolites, using liquefied sub-critical or super-critical dimethyl ether.

### BACKGROUND OF THE INVENTION

The opium poppy Papaver somniferum is an annual plant grown under International Narcotics Control Board (INCB) oversight in a number of different countries to produce alkaloids for medicinal use such as morphine, thebaine, codeine, oripavine, noscapine, papaverine and reticuline among others. The natural morphinan alkaloids (morphine, codeine, thebaine, oripavine) and benzylisoquinoline alkaloids (noscapine, papaverine and reticuline) are used as salts of their natural forms (morphine, codeine and noscapine) or as raw materials for the manufacture of semi-synthetic analogues (codeine, hydrocodone, dihydrocodeine, oxycodone, oxymorphone, hydromorphone) for the relief of severe pain (codeine, hydrocodone and dihydrocodeine), for use as anti-tussives, and as antagonists, such as naltrexone and nalmefene for the treatment of alcoholism, naloxone for use in the treatment of opiate overdose, and buprenorphine (which is a mixed agonist antagonist) for the treatment of mild to severe pain as well opiate addiction.

Extraction by water, organic solvents, or mixtures of the two, are used to extract the alkaloids from opium poppy followed by partitioning into different phases depending on the alkaloid. A number of different methods have been employed to separate and purify the individual alkaloids (Li et al., CN 102351868; An extraction method of morphine from opium: Feng et al., CN 102166432; Method for extracting morphine from opium: Tomazi, K. G., U.S. Pat. No. 7,495,098; Extraction of alkaloids from opium: Szabo et al., HU 225479; Process for the preparation and purification of solutions suitable for the extraction of opium alkaloids: Szabo et al., HU 225038; Process for separation of opium alkaloids using solvent extraction: Ma and Corcoran, U.S. Pat. No. 6,054,584; Process for extracting and purifying morphine from opium: Hodkova et al., CS 252798; Method of alkaloid isolation from opium: Durresi, S.; Revista Mjekesore (1985), (2), 98-100; and Contribution to the isolation of morphine from capsules of the domestic poppy. 1. Experimental determination of the factors which affect the process of morphine extraction: Sim S. K.; Medicinal Plant Alkaloids, 2nd Edition, Toronto Press 1970).

After extracting the alkaloids from the poppy plant, the spent (i.e., alkaloid depleted; or, having undergone alkaloid extraction) poppy plant material is useful as a key source of carbon neutral fuel. In view of the current trend toward the use of more environmentally friendly materials, the use of spent poppy material as fuel is expected to grow in the future. A disadvantage, however, of current methods for the extraction of alkaloids from opium poppies, is that the initial extraction of the alkaloids into the aqueous or aqueous/solvent mixtures leaves the spent poppy material wet with water and/or organic solvents, requiring a “drying” step before the poppy material can be used for fuel. Notably, such “drying” can be costly both financially and in terms of opportunity costs.

Another disadvantage of the current processes is the high energy costs required in the recycling of the large volumes of water or organic solvents used for extraction, along with the potential environmental losses of these solvents.

A further disadvantage of current extraction processes, especially continuous processes, relates to the presence of both water and organic solvents together in any one step of the process, resulting in the formation of emulsions which interfere with “clean” separation of phase into which alkaloids are extracted, thus, reducing the overall yield of the desired of such extracted alkaloid.

A still further disadvantage of current extraction processes, especially continuous processes, relates to the poor selectivity of the extraction using the current solvent mixtures. Typically a large amount of non-morphinan plant material is extracted into solution along with the morphinan alkaloids. During the downstream purification of the alkaloids, the presence of this non-morphinan plant material can cause the deposition of large amounts of tars. These tars can interfere with the “clean” separation of phases into which the alkaloids are extracted, thus reducing the overall yield of the desired alkaloid.

Supercritical fluids, particularly carbon dioxide and usually with polar modifiers, have been used to extract alkaloids from straw and poppy seeds, as well as to analyze the samples using super-critical fluid chromatography. A number of references (Then et al., Olaj, Szappan, Kozmetika (2000), 49 (Kulonszam), 33-39; Yoshimatsu et al., Chemical & Pharmaceutical Bulletin (2005), 53(11), 1446-1450; Janicot, J. L.; Journal of Chromatography (1990), 505(1), 247-56; Janicot, J. L., Journal of Chromatography (1988), 437(2), 351-64; Naik et al., Fluid Phase Equilibrium, 1989, 49, 115-26; Ndiomu and Simpson, Anal. Chim. Acta, 1988, 213, 237-43; and Stahl and Willing Mikrochimica Acta (1980), 2(5-6), 465-74) describe the extraction of natural substances using supercritical and liquefied gases. Fluoroform (CHF₃) was identified as “best” extractant, and carbon dioxide and nitrous oxide were approximately equivalent. However, both fluoroform, carbon dioxide and nitrous oxide are major greenhouse gases.

U.S. Pat. No. 7,696,396 discloses an extraction/enrichment procedure involving the use of liquefied or super-critical dimethyl ether to extract lipids and carotenoids from carotenoid-containing substrates. The lipids and carotenoids mentioned in the reference are either relatively non-polar or non-polar compounds, respectively. The patent, however, nowhere mentions the use of dimethyl ether in extraction processes to extract polar compounds (e.g., ionizable nitrogen containing compounds) such as morphinan and/or benzylisoquinoline alkaloids nor the use of dimethyl ether in any one step of an extraction process which contains or generates water.

Accordingly, there exists a need for a more energy efficient and environmentally friendly process. There also remains a need for methods for extracting opium poppy alkaloids which result in spent poppy material which can be immediately or directly used as fuel (e.g., without the need for “drying”). A further need remains for extraction solvents which facilitate extraction processes by reducing or eliminating the downstream formation of tars or emulsions, and which provide lower costs by more efficient solvent recovery and recycling. A further need remains for extraction solvents which preferentially extract morphinan and benzylisoquinoline alkaloids relative to non-alkaloid materials from poppy material to facilitate purification of the extracted alkaloids.

### SUMMARY OF THE INVENTION

In one embodiment, the present invention is directed to a process for the extraction of one or more of an alkaloid or a non-alkaloid material from a plant of the Papaver species comprising the steps of: (a) providing a plant of the Papaver species comprising one or more alkaloids, non-alkaloid material(s) or mixtures thereof; and (b) contacting the plant of the Papaver species with liquefied, sub-critical or super-critical dimethyl ether; to yield an extract comprising the liquefied, sub-critical or super-critical dimethyl ether and one or more alkaloid(s) and/or one or more non-alkaloid material(s).

In another embodiment, the present invention is directed to a process for the extraction of noscapine from a plant of the Papaver species comprising the steps of: (a) providing a plant of the Papaver species comprising noscapine, one or more alkaloids, non-alkaloid material(s) or mixtures thereof; and (b) contacting the plant of the Papaver species with liquefied, sub-critical or super-critical dimethyl ether; to yield an extract comprising the liquefied, sub-critical or super-critical dimethyl ether and noscapine and one or more non-alkaloid material(s).

In another embodiment, the present invention is directed to a process for the extraction of one or more alkaloid(s) and/or one or more non-alkaloid material(s) from i. a plant of the Papaver species (optionally, from the poppy straw of Papaver somniferum poppies, or optionally, dried Papaver somniferum poppies) and mutants thereof, ii. roots of Papaver bracteatum poppies, or iii. opium or tissue cultures of plant material enriched in alkaloids and/or secondary metabolites (i.e. compounds which may be derived from the alkaloids, for example, 14-hydroxycodeinone from thebaine), comprising the steps of: (a) providing a plant of the Papaver species comprising one or more alkaloid(s), one or more non-alkaloid material(s) or mixtures thereof; and (b) contacting the plant of the Papaver species with a liquefied, sub-critical or super-critical dimethyl ether, optionally, liquefied dimethyl ether, or optionally, liquefied, sub-critical dimethyl ether; to yield an extract comprising the liquefied, sub-critical or super-critical dimethyl ether (optionally liquefied, sub-critical dimethyl ether) and one or more alkaloid(s) and/or one or more non-alkaloid material(s).

In an embodiment, the present invention is directed to process for the extraction of one or more alkaloids, comprising the steps of

(a) providing a plant of the Papaver species comprising one or more alkaloids, one or more non-alkaloid material(s) or mixtures thereof;

(b) contacting the plant of the Papaver species with an organic or inorganic base; to yield a base-treated poppy plant; and

(c) contacting the base-treated poppy plant with liquefied, sub-critical or super-critical dimethyl ether; to yield an extract comprising the liquefied, sub-critical or super-critical dimethyl ether, and one or more alkaloids and/or one or more non-alkaloid material(s).

In an embodiment, the present invention is directed to a process for the extraction of one or more alkaloids, comprising the steps of

(a) providing a plant of the Papaver species comprising one or more alkaloids, one or more non-alkaloid material(s) or mixtures thereof;

(b) contacting the plant of the Papaver species with liquefied, sub-critical or super-critical dimethyl ether; to yield i) a non-alkaloid material depleted poppy plant and ii) a first extract comprising liquefied, sub-critical or super-critical dimethyl ether and one or one or more non-alkaloid material(s);

(c) contacting the non-alkaloid material depleted poppy plant with an organic or inorganic base; to yield a non-alkaloid material depleted, base-treated poppy plant;

(d) contacting the non-alkaloid material depleted, base-treated poppy plant with liquefied, sub-critical or super-critical dimethyl ether; to yield a second extract comprising the liquefied, sub-critical or super-critical dimethyl ether, water, and one or more alkaloid(s).

In another embodiment of the present invention, the alkaloids are further isolated from the above described extracts according to a process comprising the steps of:

(a) contacting an extract comprising i) a liquefied, sub-critical or super-critical dimethyl ether and ii) one or more alkaloid(s) with an aqueous base or buffer; to yield a first biphasic mixture comprising a first aqueous phase and a first organic phase; wherein the first biphasic mixture is substantially free of emulsion; and wherein the first aqueous phase of the first biphasic mixture is enriched in phenolic alkaloids; and further wherein the first organic phase of the first biphasic mixture is enriched in non-phenolic alkaloids;

(b) separating the first aqueous phase and first organic phase one from the other;

(c) isolating the phenolic alkaloids from the first aqueous phase by adjusting the pH of the first aqueous phase to a pH in the range of from about pH 7.5 to about pH 11.5 (optionally to a pH in the range of from about pH 8.5 to about pH 10.0) to form a first pH adjusted mixture; and filtering the first pH adjusted mixture;

(d) contacting the first organic phase of the first biphasic mixture with an aqueous acid; to yield a second biphasic mixture comprising a second aqueous phase and a second organic phase; wherein the second biphasic mixture is substantially free of emulsion; and wherein the second aqueous phase of the second biphasic mixture is enriched in non-phenolic alkaloids;

(e) separating the second aqueous phase and second organic phase one from the other;

(e) isolating the non-phenolic alkaloids from the second aqueous phase by adjusting the pH of the second aqueous phase to a pH in the range of from about pH 7.5 to about pH 11.5 (optionally to a pH in the range of from about pH 8.5 to about pH 10.0) to form a second pH adjusted mixture; and filtering the second pH adjusted mixture.

In another embodiment, the present invention is directed to a process for extracting and isolating alkaloids from a plant of the Papaver species comprising one or more phenolic alkaloid(s), and non-phenolic alkaloids or mixtures thereof, comprising the steps of:

(a) providing a plant of the Papaver species comprising one or more phenolic alkaloid(s), and non-phenolic alkaloids or mixtures thereof;

(b) contacting the plant of the Papaver species with liquefied, sub-critical or super-critical dimethyl ether to yield an extract comprising i) liquefied, sub-critical or super-critical dimethyl ether and ii) one or more phenolic alkaloid(s) and/or non-phenolic alkaloid(s);

(c) contacting the extract with an aqueous base or buffer; to yield a first biphasic mixture comprising a first aqueous phase and a first organic phase, wherein the first biphasic mixture is substantially free of emulsion; and wherein the first aqueous phase of the first biphasic mixture comprises one or more phenolic alkaloids such that the first aqueous phase is enriched in phenolic alkaloids and the first organic layer is substantially free of phenolic alkaloids; and further wherein the first organic phase comprises the non-phenolic alkaloids such that the first organic phase of the first biphasic mixture is enriched in non-phenolic alkaloids and the first aqueous phase is substantially free of non-phenolic alkaloids;

(d) separating the first aqueous phase and first organic phase one from the other;

(e) isolating the phenolic alkaloids from the first aqueous phase by adjusting the pH of the first aqueous phase to a pH in the range of from about pH 7.5 to about pH 11.5 (optionally to a pH in the range of from about pH 8.5 to about pH 10.0) to form phenolic alkaloid solids; and filtering the phenolic alkaloid solids from the first aqueous phase;

(f) contacting the first organic phase of the first biphasic mixture with an aqueous acid; to yield a second biphasic mixture comprising a second aqueous phase and a second organic phase; wherein the second biphasic mixture is substantially free of emulsion; and wherein the second aqueous phase of the second biphasic mixture comprises the non-phenolic alkaloids such that the second aqueous phase is enriched in non-phenolic alkaloids and the second organic phase is substantially free of non-phenolic alkaloids;

(g) separating the second aqueous phase and second organic phase one from the other; and

(h) isolating the non-phenolic alkaloids from the second aqueous phase by adjusting the pH of the second aqueous phase to a pH in the range of from about pH 7.5 to about pH 11.5 (optionally to a pH in the range of from about pH 8.5 to about pH 10.0) to form non-phenolic alkaloid solids; and filtering the non-phenolic alkaloid solids from the second aqueous phase.

In another embodiment, the present invention is directed to a process for extracting and isolating alkaloids from a plant of the Papaver species comprising one or more phenolic alkaloid(s), and non-phenolic alkaloids or mixtures thereof (for example codeine or noscapine), comprising the steps of:

(a) providing a plant of the Papaver species comprising one or more phenolic alkaloid(s), and non-phenolic alkaloids or mixtures thereof;

(b) contacting the plant of the Papaver species with liquefied, sub-critical or super-critical dimethyl ether to yield an extract comprising i) liquefied, sub-critical or super-critical dimethyl ether and ii) one or more phenolic alkaloid(s) and/or non-phenolic alkaloid(s);

(c) evaporating the liquefied, sub-critical or super-critical dimethyl ether from the extract (from step (b) above) to yield a residue comprising one or more phenolic alkaloid(s) and/or non-phenolic alkaloid(s).

In another embodiment, the present invention is directed to a process for extracting and isolating alkaloids from a plant of the Papaver species comprising one or more phenolic alkaloid(s), and non-phenolic alkaloids or mixtures thereof (for example codeine or noscapine), comprising the steps of:

(a) providing a plant of the Papaver species comprising one or more phenolic alkaloid(s), and non-phenolic alkaloids or mixtures thereof;

(b) contacting the plant of the Papaver species with liquefied, sub-critical or super-critical dimethyl ether to yield an extract comprising i) liquefied, sub-critical or super-critical dimethyl ether and ii) one or more phenolic alkaloid(s) and/or non-phenolic alkaloid(s);

(c) evaporating the liquefied, sub-critical or super-critical dimethyl ether from the extract (from step (b) above) to yield a residue comprising one or more phenolic alkaloid(s) and/or non-phenolic alkaloid(s);

(d) optionally, adding a solvent and then treating the solvent and residue mixture with an acid; to yield the corresponding acid addition salt of the phenolic and/or non-phenolic alkaloid(s) which may, optionally, be isolated by filtration.

In an example, to the residue (of step (c) above) is optionally added a water miscible organic solvent (such as ethanol, methanol, propanol, isopropanol, n-butanol, and the like) followed by a suitably selected acid such as HCl, HBr, tartaric acid, sulfuric acid, phosphoric acid, acetic acid, formic acid, and the like; to yield a precipitate; wherein the precipitate comprises the acid addition salt of the phenolic or non-phenolic alkaloid(s).

### BRIEF DESCRIPTION OF THE FIGURES

FIG. 1 is a flow-chart illustrating an example of an embodiment of the one step process of the present invention for extracting and isolating alkaloids from poppy straw (an example of a plant of the Papaver species).

FIG. 2 is a flow-chart illustrating a second embodiment of the one step process of the present invention for extracting and isolating alkaloids from poppy straw (an example of a plant of the Papaver species).

FIG. 3 is a flow-chart illustrating an embodiment of the two step process of the present invention for extracting and isolating alkaloids from poppy straw (an example of a plant of the Papaver species).

FIG. 4 is a flow chart illustrating a second embodiment of the two step process of the present invention for extracting and isolating alkaloids from poppy straw (an example of a plant of the Papaver species).

FIG. 5 is a schematic illustrating the laboratory scale extraction system used in certain of the Examples which follow herein.

FIG. 6 illustrates the retention times for a standard solution of alkaloids, as measured according to the procedure described in the Examples section which follows herein.

### DETAILED DESCRIPTION OF THE INVENTION

In one embodiment, the present invention is directed to a method for the extraction of one or more alkaloids and/or one or more non-alkaloid material(s) from a plant of the Papaver species, comprising contacting the plant of the Papaver species with liquefied, sub-critical or super-critical dimethyl ether, optionally, liquefied, sub-critical dimethyl ether, to yield an extract comprising i) the liquefied, sub-critical or super-critical dimethyl ether and ii) the extracted alkaloid(s) and/or non-alkaloid material(s), as described in more detail herein.

In certain embodiments, the plant of the Papaver species is selected from the group consisting of (a) poppy straw resulting from the end of the growth cycle of the Papaver somniferum poppy, or mutants thereof, (b) poppy straw resulting from the end of the growth cycle of a thebaine-rich mutant of the Papaver somniferum poppy, (c) poppy straw resulting from the end of the growth cycle of a codeine-rich mutant of the Papaver somniferum poppy, (d) poppy straw resulting from the end of the growth cycle of a oripavine and thebaine-rich mutant of the Papaver somniferum poppy, (e) poppy straw resulting from the end of the growth cycle of morphine-rich Papaver sominferum poppy, (f) poppy straw resulting from the end of the growth cycle of a noscapine-rich mutant of the Papaver somniferum poppy, (g) the roots of the Papaver bracteatum poppy, (h) opium or extracts thereof, and (i) tissue cultures of plant material enriched in secondary metabolites or mixtures thereof.

In another embodiment of the present invention, the plant of the Papaver species is selected from the group consisting of the poppy straw of the top 10 centimeters of the Papaver somniferum poppy or mutants thereof, and the roots of the Papaver bracteatum poppy. In another embodiment of the present invention, the plant of the Papaver species is selected from the group consisting of poppy straw resulting from the end of the growth cycle of a thebaine-rich mutant of the Papaver somniferum poppy, poppy straw resulting from the end of the growth cycle of a codeine-rich mutant of the Papaver somniferum poppy, poppy straw resulting from the end of the growth cycle of a oripavine and thebaine-rich mutant of the Papaver somniferum poppy, poppy straw resulting from the end of the growth cycle of the morphine-rich Papaver somniferum poppy and poppy straw resulting from the end of the growth cycle of the noscapine-rich Papaver somniferum poppy.

In an embodiment, the present invention is directed to processes for the extraction of thebaine from thebaine-rich poppy straw (for example, from the mutant Papaver somniferum poppy described in US 20090227796, herein incorporated by reference in its entirety). In another embodiment, the present invention is directed to processes for the extraction of codeine and thebaine from a poppy straw rich in codeine and thebaine (for example, from the poppy mutant described in US Patent Publication 2010/0234600, herein incorporated by reference in its entirety). In another embodiment, the present invention is directed to processes for the extraction of thebaine and oripavine from a poppy straw rich in thebaine and oripavine (for example, from the poppy mutant described in U.S. Pat. No. 6,067,749, U.S. Pat. No. 6,376,221 and U.S. Pat. No. 6,723,894, each of which patents are herein incorporated by reference in their entirety). In another embodiment, the present invention is directed to processes for the extraction of morphine from a poppy straw rich in morphine (for example, from a standard Papaver somniferum poppy). In another embodiment, the present invention is directed to processes for the extraction of noscapine from a poppy straw containing or rich in noscapine (e.g., high producing poppy plants described in WO2013136057 to Winzer et al., filed Mar. 12, 2013, herein incorporated by reference).

In an embodiment of the present invention, the plant of the Papaver species is pre-treated with an organic or inorganic base, optionally, an aqueous inorganic base, optionally KOH, NaOH, an aqueous calcium hydroxide, and the like, optionally, KOH or a slurry of 2% w/w aqueous calcium hydroxide, prior to contacting the plant of the Papaver species with the liquefied, sub-critical or super-critical dimethyl ether (optionally, liquefied, sub-critical dimethyl ether). In another embodiment of the present invention, the plant of the Papaver species is not pre-treated with an organic or inorganic base prior to contacting the plant of the Papaver species with the liquefied, sub-critical or super-critical dimethyl ether (optionally, liquefied, sub-critical dimethyl ether).

In another embodiment, the present invention is directed to processes for the isolation of any one or any mixture of one or more alkaloids (including morphinan and/or benzylisoquinoline alkaloids), as described in more detail herein.

In an embodiment, the present invention is directed to methods for the extraction of alkaloids (e.g., one or more morphinan or benzylisoquinoline alkaloid(s)) and/or one or more non-alkaloid material(s) from a plant of the Papaver species, comprising contacting the plant of the Papaver species with liquefied, sub-critical or super-critical dimethyl ether, optionally, liquefied, sub-critical dimethyl ether, to yield an extract comprising i) the liquefied, sub-critical or super-critical dimethyl ether and ii) the extracted alkaloid(s) and/or non-alkaloid material(s), as described in more detail herein, wherein the extraction process steps are, each independently, run under batch or continuous process conditions.

In another embodiment, the present invention is directed to methods for the extraction of alkaloids (e.g., one or more morphinan or benzylisoquinoline alkaloid(s)) and/or one or more non-alkaloid material(s) from a plant of the Papaver species, comprising contacting the plant of the Papaver species with liquefied, sub-critical or super-critical dimethyl ether, optionally, liquefied, sub-critical dimethyl ether, to yield an extract comprising i) the liquefied, sub-critical or super-critical dimethyl ether and ii) the extracted alkaloid(s) and/or non-alkaloid material(s), as described in more detail herein, wherein one or more of the process steps are run under continuous process conditions.

In an embodiment, the present invention is directed to processes for the extraction of any of one or more morphinan alkaloids from the straws of (a) Papaver somniferum poppy mutants (e.g. codeine and thebaine from the poppy mutant rich in codeine; oripavine and thebaine from the poppy mutant rich in oripavine and thebaine, and thebaine from the poppy rich in thebaine, etc.), (b) morphine Papaver somniferum poppy, (c) Papaver bracteatum poppy (e.g. thebaine from the roots of said Papaver bracteatum poppy), (c) opium or mixtures or extracts of opium, and (d) tissue cultures of poppy plant materials enriched in secondary metabolites according to the extraction processes of the present invention.

In another embodiment, the present invention is directed to processes for the extraction of any one or more benzylisoquinoline alkaloids from the straws of (a) Papaver somniferum poppy mutants (e.g. noscapine, etc.), (b) morphine Papaver somniferum poppy, (c) opium or mixtures or extracts of opium, and (d) tissue cultures of poppy plant materials enriched in secondary metabolites according to the extraction processes of the present invention.

In another embodiment, the present invention is directed to a process for the extraction of thebaine from thebaine-rich poppy straw, (wherein the poppy straw is prepared for extraction according to known processes, for example by harvesting, drying, threshing to form a straw, milling the straw to a small particle size and then treating with an aqueous organic or inorganic base) according to the extraction processes of the present invention. In another embodiment, the present invention is directed to a process for the extraction of codeine and thebaine from the codeine-rich poppy according to the extraction processes of the present invention. In another embodiment, the present invention is directed to a process for the extraction of oripavine and thebaine from oripavine-rich and thebaine-rich poppy according to the extraction processes of the present invention. In another embodiment, the present invention is directed to a process for the extraction of morphine from morphine-rich poppy according to the extraction processes of the present invention. In another embodiment, the present invention is directed to a process for the extraction of noscapine from noscapine-rich poppy according to the extraction processes of the present invention.

In an embodiment of the present invention, the process for the isolation of any one or any mixture of one or more alkaloids comprises the step of formation of a biphasic mixture, wherein the biphasic mixture is substantially free of emulsion.

In another embodiment of the present invention, the process for the isolation of any one or any mixture of one or more alkaloids comprises the step of formation of a biphasic mixture, wherein the biphasic mixture is substantially free of emulsion; and wherein one or more steps in the isolation process is run under continuous process conditions.

In another embodiment of the present invention, the process for the isolation of any one or any mixture of one or more alkaloids comprises the step of formation of an extract comprising the liquefied, sub-critical or super-critical dimethyl ether and said one or mixture of one or more alkaloids (in solution); and wherein the extract comprises total dissolved solids which are rich in (or enriched in) one or more alkaloid(s) relative to non-alkaloid materials.

In another embodiment of the present invention, the process for the isolation of any one or any mixture of one or more alkaloids comprises the step of formation of an extract comprising the liquefied, sub-critical or super-critical dimethyl ether and said one or mixture of one or more alkaloids (in solution); wherein the extract is subjected to evaporation to remove the dimethyl ether and isolate the dissolved solids as a residue; wherein the isolated solids are rich in (or enriched in) one or more alkaloid(s) relative to non-alkaloid materials.

In certain embodiments of the present invention, the processes described herein are run under batch process conditions. In certain other embodiment of the present invention, one or more independently selected steps of any of the processes described herein are run under continuous process conditions. In another embodiment of the present invention, the isolation of one or more alkaloids (morphinan and/or benzylisoquinoline alkaloids) is completed under continuous process conditions.

### Definitions

The processes of the present invention can comprise, consist of, or consist essentially of the steps, essential elements and limitations of the invention described herein, as well as any of the additional or optional ingredients, components, or limitations described herein. The term “comprising” (and its grammatical variations) as used herein is used in the inclusive sense of “having” or “including” and not in the exclusive sense of “consisting only of.”

The terms “a” and “the” as used herein are understood to encompass the plural as well as the singular.

As used herein, unless otherwise noted, the term “C₁₋₄ alkyl” shall include straight and branched chain compositions of between 1 and 4 carbon atoms including methyl, ethyl, n-propyl, isopropyl, n-butyl, isobutyl and t-butyl. In an embodiment, the C₁₋₄ alkyl is selected from the group consisting of methyl, ethyl and isopropyl.

As used herein, unless otherwise noted, the term “C₁₋₄alkyl alcohol” shall mean any C₁₋₄alkyl chain substituted with at least one (optionally one) hydroxy (—OH) group. Suitably examples include, but are not limited to methanol, ethanol, propanol, isopropanol, n-butanol, t-butanol, and the like. In an embodiment, the C₁₋₄alkyl alcohol is selected from the group consisting of methanol, ethanol and isopropanol, optionally methanol or ethanol.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed description which follows herein. One skilled in the art will recognize that the listing of said examples is not intended, and should not be construed, as limiting in any way the invention set forth in the claims which follow thereafter.

Unless otherwise indicated, all documents cited are, in relevant part, incorporated herein by reference; the citation of any document is not to be construed as an admission that it is prior art with response to the present invention. Furthermore, all documents incorporated herein by reference in their entirety are only incorporated herein to the extent that they are not inconsistent with this specification.

The present invention is directed to processes for the extraction of one or more morphinan and/or one or more benzylisoquinoline alkaloids and/or one or more non-alkaloid materials from a plant of the Papaver species (for example from poppy straw of Papaver somniferum poppy (optionally, dried Papaver somniferum poppy), or mutants thereof) according to the extraction processes of the present invention. The present invention is further directed to processes for the extraction of one or more morphinan and/or one or more benzylisoquinoline alkaloids and/or one or more non-alkaloid materials from the roots of Papaver bracteatum poppy according to the extraction processes of the present invention.

As used herein, unless otherwise noted, the terms “plant of the Papaver species” and “poppy plant” shall mean any mixture of plant materials derived from a plant of the Papaver species, which material contains one or more morphinan alkaloids and/or one or more benzylisoquinoline alkaloids. Said material may include any mixture of seed capsules, bulbs, seeds, roots, stems, leaves or other component of the poppy plant. In an embodiment of the present invention, the plant of the Papaver species is the top 10 centimeters of Papaver somniferum (optionally, dried Papaver somniferum) or mutants thereof. In another embodiment of the present invention, the plant of the Papaver species is roots of the Papaver bracteatum poppy.

As used herein, unless otherwise noted, the term “poppy straw” shall mean any mixture of plant material of the Papaver somniferum poppy (optionally, dried Papaver somniferum) including but not limited to the seed capsules, bulbs, seeds, roots, stems, leaves or other component of the poppy plant. In an embodiment, the poppy straw comprises the top 10 cm of the plant, including the capsules and stems of Papaver somniferum poppy (optionally, dried Papaver somniferum). In certain embodiments, the poppy straw is dried poppy straw.

As used herein, unless otherwise noted, the term “morphinan alkaloid” shall mean any compound containing the base chemical structure (I), also known as the morphinan ring structure:

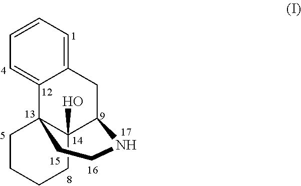

Suitably examples include any one or mixture of one or more of thebaine, codeine, oripavine and morphine. One skilled in the art will recognize that morphinan alkaloids are polar compounds having high molecular weight (molecular weight greater than about 250 g/mol).

As used herein, unless otherwise noted, the term “benzylisoquinoline alkaloid” shall mean any compound containing the base chemical structure (II), also known as the benzylisoquinoline ring structure:

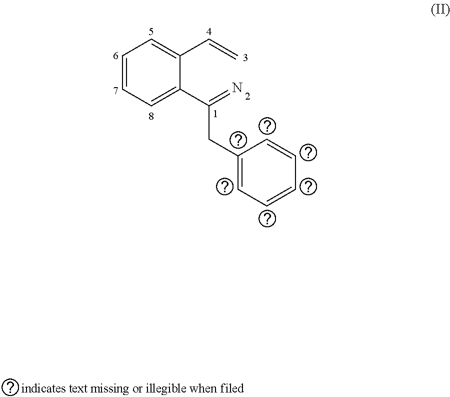 [SMILES: C=Cc1ccccc1C(=N)Cc1ccccc1]

Suitable examples include any one or mixture of one or more of noscapine, papaverine and reticuline. One skilled in the art will recognize that benzylisoquinoline alkaloids are polar compounds having high molecular weight (molecular weight greater than about 250 g/mol).

One skilled in the art will recognize that polar compounds (such as the morphinan and benzylisoquinoline alkaloids described herein) are compounds which exhibit polarity (characterized by having two opposing charges or poles). Thus, polar compounds are soluble in water (which is a polar solvent) and on dissolving in water are present as an ionic form (i.e., as an ion). Additionally, polar compounds are molecules which are linked through chemical bonds arranged such that the distribution of charges is unsymmetrical. One skilled in the art will further recognize that the morphinan and benzylisoquinoline alkaloids comprise an ionizable nitrogen atom, which contributes to the polarity of said compounds. By contrast, non-polar compounds (such as carotenoids and lipids, which, absent indication otherwise, do not comprise an ionizable nitrogen) do not exhibit polarity, do not convert into ions in solution, do not readily dissolve in water and do not comprise an unsymmetrical distribution of charges.

As used herein, unless otherwise noted, the terms “alkaloid-rich” or “alkaloid enriched” when referring to a poppy plant, (wherein the alkaloid is a morphinan or benzylisoquinoline alkaloids as herein described, for example, morphine, codeine, thebaine, oripavine, noscapine, etc.) shall mean a poppy plant wherein the named alkaloid is present, or a poppy plant that is engineered so that the named alkaloid is present, in an amount constituting 50% (or about 50%), optionally, 60% (or about 60%), optionally, 70% (or about 70%), optionally, 80% (or about 80%), by weight, or greater of the sum total of all the alkaloids present in the poppy plant. Optionally, the terms “alkaloid-rich” or “alkaloid enriched” when referring to a poppy plant, (wherein the alkaloid is a morphinan or benzylisoquinoline alkaloids as herein described, for example, morphine, codeine, thebaine, oripavine, noscapine, etc.) shall mean a poppy plant wherein the named alkaloid is present, or a poppy plant that is engineered so that the named alkaloid is present, in an amount constituting 50% (or about 50%), optionally, 60% (or about 60%), optionally, 70% (or about 70%), optionally, 80% (or about 80%), by weight, or greater of the sum total of codeine, morphine, thebaine and orapavine alkaloids present in the poppy plant.

As used herein, unless otherwise noted, the terms “morphinan and/or benzylisoquinoline alkaloid containing poppy plant” and “alkaloid containing poppy plant” shall mean a plant or mixture of plants of the Papaver species (as herein defined) which contains one or more morphinan and/or one or more benzylisoquinoline alkaloids. In certain embodiments, the alkaloid containing poppy plants include, but are not limited to, poppy straw from Papaver somniferum poppies (optionally, dried Papaver somniferum), and mutants thereof, roots of Papaver bracteatum poppies, opium and tissue cultures of plant material enriched in alkaloids and/or secondary metabolites or mixtures thereof. One skilled in the art will recognize that the alkaloid containing poppy plant may further contain “non-alkaloid materials” which are, for example, non-polar components such as lipids, tars, waxes, and other naturally occurring non-polar metabolites (such as for example proteins, fatty acids (linoleic, palmitic, oleic), lipids, triglycerides, flavone/isoflavone, flavanone and flavanoid/isoflavonoid, cellulose and high molecular weight vegetable matter, etc.).

In one embodiment, the morphinan and/or benzylisoquinoline alkaloid containing poppy plant is poppy straw derived from the uppermost 10 centimeters of the Papaver somniferum poppy which includes the seed capsule (optionally, dried seed capsule). This portion of the plant contains over 90% of the alkaloids produced during the growth period of the plant. In another embodiment, the Papaver somniferum poppy mutants include, but are not limited to thebaine-rich poppy, codeine-rich poppy, oripavine-rich and thebaine-rich poppies, morphine Papaver somniferum poppy, noscapine-rich poppies, reticuline-rich poppies and other mutants yet to be developed, and the like.

As used herein, unless otherwise noted, the terms “base treated poppy plant” and “base pre-treated poppy plant” shall mean any alkaloid containing poppy plant, as herein defined, wherein the alkaloid containing poppy plant has been treated with a suitably selected organic base (e.g. triethylamine) or inorganic base, optionally dissolved or suspended in a small amount of water or suitably selected solvent (e.g. a low molecular weight alcohol, optionally a C₁₋₄alkyl alcohol). Suitable examples of said bases include, but are not limited to, an inorganic base such as KOH, NaOH, calcium hydroxide, ammonia, potassium carbonate, sodium carbonate, sodium bicarbonate and the like or mixtures thereof, optionally, dissolved or slurried in water. In certain embodiments, the suitably selected organic or inorganic base is an inorganic base. In other embodiments, the suitably selected organic or inorganic base is an aqueous inorganic base. In other embodiments, the aqueous inorganic base is a slurry of 2% w/w aqueous calcium hydroxide in water. In still further embodiments, the suitably selected organic or inorganic base is an inorganic base dissolved or slurried in a small volume of a low molecular weight alcohol, for example a C₁₋₄alkyl alcohol, such as methanol, ethanol, isopropanol, and the like, optionally methanol or ethanol. One skilled in the art will recognize that in the base treated poppy plant, the morphinan and/or benzylisoquinoline alkaloid(s) have been liberated from the cellular matrix of the poppy plant.

As used herein, unless otherwise noted, the term “non-alkaloid material depleted poppy plant” shall mean any alkaloid containing poppy plant which has been treated (for example, extracted with liquefied, sub-critical or super-critical dimethyl ether, optionally liquefied, sub-critical dimethyl ether) to remove a substantial amount of non-alkaloid materials present in the poppy plant, optionally greater than about 50% of the total extractable non-alkaloid material present in the poppy plant, optionally, greater than about 60%, optionally, greater than about 70%, optionally, greater than about 80%, or, optionally greater than about 90%.

As used herein, unless otherwise noted, the terms “extract” and “total extract” shall mean any composition or mixture resulting from an extraction process using liquefied, sub-critical or super-critical dimethyl ether (optionally liquefied, sub-critical dimethyl ether), comprising i) the liquefied, sub-critical or super-critical dimethyl ether (optionally liquefied, sub-critical dimethyl ether) and ii) one or more alkaloid(s); one or more non-alkaloid material(s) or mixtures thereof. In certain embodiment, the extract or total extract may additionally comprise water.

As used herein, unless otherwise noted, the term “super-critical” shall mean, with respect to the dimethyl ether, a gaseous or liquid dimethyl ether that is above both its critical temperature and critical pressure.

As used herein, unless otherwise noted, the term “super-critical fluid” shall mean a gas or liquid that is present at above both its critical temperature and critical pressure. As used herein, the term “super-critical fluid” shall include individual solvents and mixture of solvents. Wherein the “super-critical fluid” is a mixture of solvents, the super-critical fluid is present at above both the critical temperature and critical pressure of the mixture.

As used herein, unless otherwise noted, the term “sub-critical” shall mean, with respect to the dimethyl ether, a gaseous or liquid dimethyl ether that is below its critical temperature and critical pressure. As used herein, unless otherwise noted, the term “sub-critical fluid” shall mean a gas or liquid that is below its critical temperature and critical pressure. As used herein, the term “sub-critical fluid” shall include individual solvents and mixture of solvents.

As used herein, unless otherwise noted, the term “liquefied” shall mean, with respect to the dimethyl ether, dimethyl ether which exists as a gas under ambient temperatures and pressures, which is below its critical temperature and which is compressed to a point above its vapor pressure (which may or may not be above its critical pressure), so as to yield a liquid. One skilled in the art will recognize that dimethylethyl may also be liquefied at its sub-critical or super-critical temperature and/or pressure.

As used herein, unless otherwise noted, the term “liquefied gas” shall mean a compound which exists as a gas under ambient temperatures and pressures, which is below its critical temperature and which is compressed to a point above its vapor pressure (which may or may not be above its critical pressure), so as to yield a liquid. One skilled in the art will recognize that a liquefied solvent may also be present at sub-critical or super-critical temperature and/or pressure.

Suitable exemplary solvents (and co-solvents) useful as supercritical fluids, subcritical fluids or liquefied gases include, but are not limited to: carbon dioxide (CO₂), nitrous oxide (N₂O), toluene and mixtures thereof.

Ethers (such as liquid ethers such as diethyl ether) are generally good solvents for dissolving many organic materials. Ethers dissolve a wide range of polar and non-polar substances, and are also good solvents for many hydrogen-bonded substances (e.g. water). Hydrogen-bonded substances need more solvation energy to break the hydrogen bonds that hold such molecules together and ethers can act as hydrogen-bond acceptors, forming hydrogen bonds with hydrogen-bonding solutes. Ethers also have relatively low boiling points, and are relatively easily evaporated from products. In an example, diethylether is used in the pharmaceutical industry as an extraction solvent; however, the potential build-up of explosive peroxides limits its use.

As noted above, while carbon dioxide can be used as a super-critical fluid for the extraction of organic materials, it is not desirable for use in large scale manufacture because, for example, it is a greenhouse gas, and is not environmentally friendly, particularly in processes which would release large volumes into the atmosphere.

Dimethyl ether has a solubility in water of 70 grams/Liter. When large volumes of pressurized dimethyl ether gas are used in the presence of water, a dimethyl ether/water single phase solution forms. This property allows the formation of a more polar solution for the extraction of, for example, highly polar morphinan and benzylisoquinoline alkaloids. The water in such a dimethyl ether/water single phase solution can be provoked to form a separate phase by the addition of an inorganic base, acid or salt. This property allows for the separation of phenolic from non-phenolic alkaloids and also for the separation of alkaloids having different pKa values. The particularly low boiling point of dimethyl ether, −25° C., is additionally advantageous as it is a gas at atmospheric pressure and ambient temperature. Therefore, products extracted with dimethyl ether are produced substantially solvent free. However, dimethyl ether is an ether which is not generally employed as an industrial solvent, since it must be compressed to a liquid state or to a supercritical state to become a solvent. The critical temperature and pressure for dimethyl ether are 127.2° C. and 773 psi (˜52 atm). Finally, it is notable (and advantageous, particularly for processes which would use large amounts or volumes of dimethyl ether) that dimethyl ether (unlike other ethers such as ethylether) does not form peroxides and is not currently identified as a greenhouse gas.

In an embodiment, the present invention is directed to the use of liquefied, sub-critical or super-critical dimethyl ether for the extraction of one or more morphinan and/or one or more benzylisoquinoline alkaloids from alkaloid containing poppy plant according to the processes of the present invention, optionally, substantially free of an emulsion during the extraction process.

In another embodiment, the present invention is directed to the use of liquefied, sub-critical or super-critical dimethyl ether for the extraction of one or more non-alkaloid materials from alkaloid containing poppy plant according to the processes of the present invention, resulting in an alkaloid containing non-alkaloid material depleted poppy plant, optionally, wherein any extract obtained during the extraction process is substantially free of an emulsion; followed by base treatment of the alkaloid containing non-alkaloid material depleted poppy plant and subsequent extraction of the base treated, alkaloid containing non-alkaloid material depleted poppy plant with liquefied, sub-critical or super-critical dimethyl ether for the extraction of alkaloids, namely one or more morphinan and/or one or more benzylisoquinoline alkaloids, optionally wherein any extract obtained during the extraction process is substantially free of an emulsion.

As outlined in the Compendium of Chemical Terminology, 2nd ed. (the “Gold Book”). Compiled by A. D. McNaught and A. Wilkinson, Blackwell Scientific Publications, Oxford, UK (1997), emulsions can be detected in a water/water-insoluble organic solvent mixture because they have different physical properties relative to both the water and the water-insoluble organic solvent phases. For example, emulsions tend to have a cloudy appearance because the many phase interfaces scatter light in the visible spectrum as it passes through the emulsion, provided the droplet sizes exceed about one-quarter of the wavelength of the incident light. Emulsions appear white when all light is scattered equally. If the emulsion is dilute enough, higher-frequency and low-wavelength light will be scattered more, and the emulsion will appear bluer—this is called the “Tyndall effect”. If the emulsion is concentrated enough, the color will be distorted toward comparatively longer wavelengths, and will appear more yellow. Similarly, the electrical conductivity of an emulsion will be measurably different from both the water and the water-insoluble organic solvent phases. Techniques to detect and measure emulsions at the interface of both the water and the water-insoluble organic solvent phases are well known in the art and include:
Visual inspection for turbidity; the continuous water and the water-insoluble organic solvent phases are usually clear while emulsions are generally turbid;
Color monitoring; and
Electrical conductivity probes.

As used herein, unless otherwise noted, the term “substantially free of an emulsion” when referring to a biphasic mixture in an extraction process shall refer to a biphasic mixture which exhibits two distinct phases, as determined, for example, by the visual inspection, such that greater than about 90% wt/wt, optionally greater than about 95% wt/wt, or optionally greater than 99% wt/wt of the total biphasic mixture is present in the two separated, phases. One skilled in the art will recognize that wherein the biphasic mixture is substantially free of an emulsion, said biphasic mixture can be cleanly separated, under batch and advantageously, under continuous processing conditions.

As used herein, the term “cleanly separated” when referring to the separation of phases of the biphasic mixture, means that greater than about 90%, optionally greater than about 95%, optionally greater than 99% of one phase of the biphasic mixture may be removed from the other phase such that the removed phase is free of or substantially free of the other phase. The term “substantially free of the other phase” for purposes of this definition means less than about 5%, optionally less than about 3%, optionally less than about 1%, optionally less than about 0.01%, by weight, of the remaining phase is present in the removed phase.

As used herein, the term “visual inspection” means that a human viewer can visually discern the presence of turbidity with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 0.25 meter. Visual inspection of a continuous process extraction system can be facilitated by incorporating into the extraction system a sight glass suitable for the pressure used in such extraction system/process.

As used herein, unless otherwise noted, the term “substantially free”, when referring to the presence of phenolic or non-phenolic alkaloid remaining in an aqueous or organic phase from which it was extracted, means that the phase from which the phenolic or non-phenolic alkaloid was extracted has remaining therein less than about 25%, optionally less than about 10%, optionally less than about 5%, optionally less than about 2%, optionally less than about 1%, optionally less than about 0.5%, or optionally less than about 0.1%, by weight, of the extracted phenolic or non-phenolic alkaloid, as the case may be, by weight of the total weight of such phase and the remaining phenolic or non-phenolic alkaloid, as the case may be. For example, wherein a total extract containing phenolic and non-phenolic alkaloids is further extracted with an aqueous base to yield an aqueous and organic phase biphasic mixture such that the aqueous phase of the biphasic mixture comprises phenolic alkaloids and is substantially free of non-phenolic alkaloids (as substantially all the non-phenolic alkaloids remain unextracted in the organic phase), said aqueous phase, therefore, contains less than about 25 wt % (optionally less than about 10 wt %, optionally less than about 5 wt %, optionally less than about 2 wt %, optionally less than about 1 wt %) of the total amount of the aqueous phase and the remaining non-phenolic alkaloid.

As used herein, the term “difficultly soluble” as used herein means, with respect to the solubility of aqueous acid in dimethyl ether, a solubility of less than or equal to 1%, optionally less than 0.1%, optionally less than 0.01%, by weight, of the aqueous acid in dimethyl ether at 25° C.

As to the mechanism of the extraction or the identity of any critical parameters or critical properties of the components of the extraction processes of the present invention, yet without being limited by theory, it is believed that liquefied, sub-critical or super-critical dimethyl ether yields biphasic mixtures which are substantially free of emulsion as a result of the low density of dimethyl ether (0.67 g/mL at 20° C.) and/or due to the slight solubility of water in dimethyl ether (˜5% wt/wt at 20° C.).

Advantageously, the processes of the present invention preferentially extract alkaloids from poppy species such that the isolated extract(s) and/or residues are enriched in extracted alkaloid(s) relative to non-alkaloid material(s).

The present invention is directed to processes for the extraction of one or more morphinan and/or benzylisoquinoline alkaloid(s) (and/or one or more non-alkaloid material(s)), as described in more detail below. According to the present invention, the morphinan and/or benzylisoquinoline alkaloid(s) are extracted from an alkaloid-containing poppy plant by contacting said poppy plant with liquefied, sub-critical or super-critical dimethyl ether, optionally liquefied dimethyl ether, or optionally liquefied, sub-critical dimethyl ether.

Accordingly, in an embodiment of the present invention, an alkaloid containing poppy plant (e.g. a morphinan and/or benzylisoquinoline alkaloid containing poppy plant) is initially charged into a suitable batch or continuous (process) extraction vessel. In an embodiment of the present invention, the alkaloid containing poppy plant is poppy straw. In another embodiment of the present invention, the alkaloid containing poppy plant is base-treated poppy plant.

The alkaloid containing poppy plant may be charged to the extraction vessel in any form which allows for handling, optionally in a form which allows for handling on a large scale/manufacture/production scale. In an embodiment, the alkaloid containing poppy plant is charged to the extraction vessel as a solid powder of milled straw or crushed roots. In an embodiment,the bulk density of the solid powder of milled straw is in range of from about 0.2 g/cc to about 0.25 g/cc, or any amount or range therein. One having ordinary skill in the art will understand, however, that the present invention is in no way limited to solid feeds or to solid feeds of this range of bulk density, since liquid alkaloid containing poppy extracts (for example, opium in a solution of water at an apropriate pH, as would be readily know to those skilled in the art) may also be used.

One skilled in the art will further recognize that the amount of alkaloid containing poppy plant charged into the extraction vessel is directly related to the volume of the extraction vessel used in the extraction process. For example, if a 1-liter extraction vessel is used to extract a solid powder of milled straw with a bulk density of about 0.2 g/cc, then approximately 200 grams of the solid powder of milled straw will be charged to the vessel.

The extraction vessel is then sealed, and the temperature and pressure within the extraction vessel are adjusted to maintain the extracting fluid (for example, dimethyl ether) in liquid form, optionally adjusted to a temperature in the range of from about 0° C. to about 100° C., optionally to a temperature in the range of from about 25° C. to about 75° C., or optionally to a temperature in the range of from about 45° C. to about 55° C., or, optionally, any temperature or range of temperatures therein; and a pressure, optionally, adjusted to a pressure in the range of from about 4 atm to about 25 atm, optionally, to a pressure in the range of from about 12 atm to about 18 atm, or, optionally, any pressure or range of pressures therein.

Alternatively, the extraction vessel is then sealed, and the temperature and pressure within the extraction vessel are adjusted to maintain the extracting fluid (for example, dimethyl ether) as super-critical fluid, optionally, adjusted to a temperature in the range of from about 0° C. to about 100° C., optionally, to a temperature in the range of from about 30° C. to about 70° C., optionally, to a temperature in the range of from about 45° C. to about 55° C., or, optionally, any temperature or range of temperatures therein; and a pressure, optionally, adjusted to a pressure in the range of from about 4 atm to about 35 atm, optionally, to a pressure in the range of from about 10 atm to about 20 atm, or, optionally, any pressure or range of pressures therein.

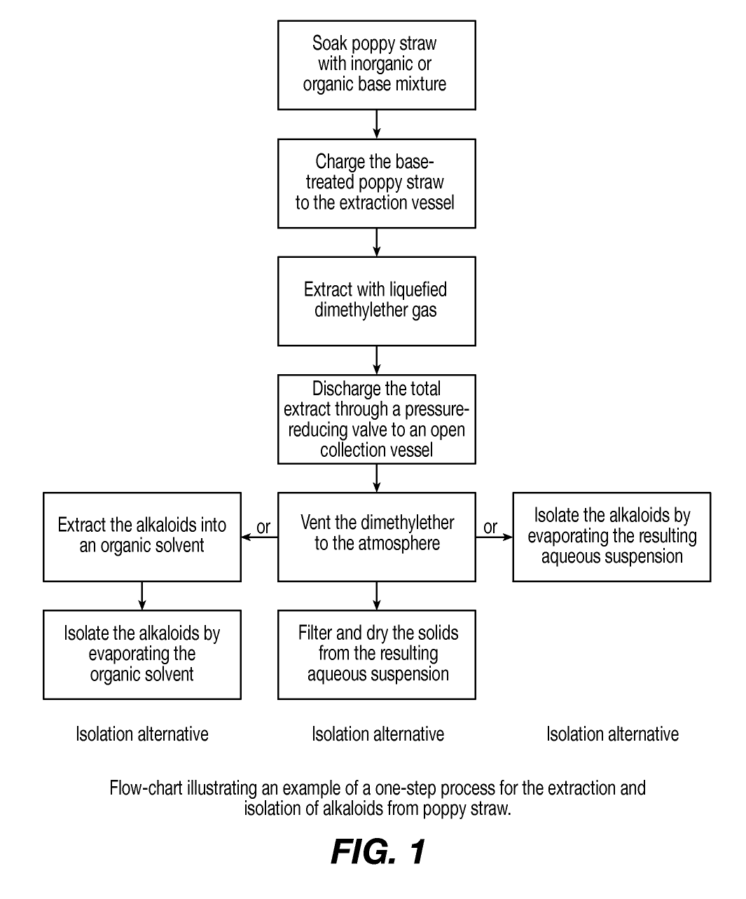

In an embodiment, the present invention is directed to a first extraction method, as illustrated in FIG. 1, wherein base treated poppy plant containing non-alkaloids and alkaloids (i.e., morphinan and/or benzylisoquinoline alkaloid(s)) (for example base treated poppy straw as exemplified in FIG. 1) is charged to the extracted vessel and then contacted with liquefied, sub-critical or super-critical dimethyl ether, optionally liquefied dimethyl ether, optionally, liquefied, sub-critical dimethyl ether; to yield a total extract comprising i) the liquefied, sub-critical or super-critical dimethyl ether, ii) water, and iii) the extracted alkaloid(s) (optionally, such that the extracted alkaloids are dissolved in a mixture of liquefied, sub-critical or super-critical dimethyl ether and water). One skilled in the art will recognize that in addition to the morphinan and/or benzylisoquinoline alkaloid(s), the liquefied, sub-critical fluid or super-critical dimethyl ether; water or mixture thereof further contains extracted non-alkaloid materials including, but not limited to lipids, waxes, tars, peptides, other plant metabolites, etc.

The total extract is then further processed or treated, according to known methods, to further isolate the morphinan and/or benzylisoquinoline alkaloid(s) as a residue, optionally, as a solid. In an example, the extract is vented to release (e.g. evaporate) the pressurized liquefied, sub-critical or super-critical dimethyl ether (optionally, liquefied dimethyl ether, or, optionally, liquefied, sub-critical dimethyl ether), and yield an aqueous suspension of the morphinan and/or benzylisoquinoline alkaloid(s) (along with any extracted non-alkaloid materials such as lipids, tars, waxes, etc.). The morphinan and/or benzylisoquinoline alkaloid(s)) are then isolated from the aqueous mixture according to known methods, for example by filtration or evaporation of the aqueous phase. In another example, the total extract is extracted with a suitably selected organic solvent such as toluene, n-butanol, tetrahydrofuran, methyltetrahydrofuran, and the like, optionally toluene; to yield a biphasic mixture (optionally substantially free of emulsion), wherein the morphinan and/or benzylisoquinoline alkaloids are partitioned between the organic and aqueous phases depending on differences in their physico-chemical properties (solubility, pK_{b} etc); and the morphinan and/or benzylisoquinoline alkaloid(s) are then isolated from the organic and aqueous phases according to known methods, for example by crystallization, filtration or evaporation of the organic solvent.

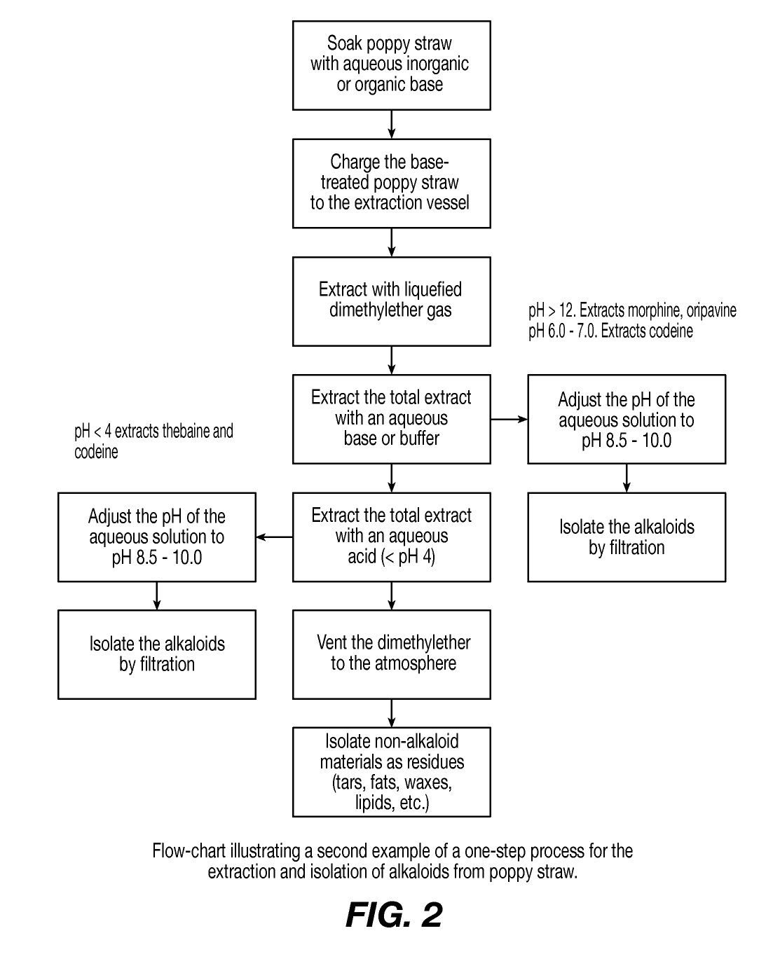

In another embodiment, the present invention is directed to a second extraction method, as illustrated in FIG. 2, wherein base treated poppy plant containing non-alkaloids and alkaloids (i.e., morphinan and/or benzylisoquinoline alkaloid(s), including phenolic and nonphenolic alkaloids) (for example base treated poppy straw as exemplified in FIG. 2) is charged to the extracted vessel and then contacted with liquefied, sub-critical or super-critical dimethyl ether (optionally liquefied dimethyl ether,or optionally liquefied, sub-critical dimethyl ether); to yield a total extract comprising i) the liquefied, sub-critical or super-critical dimethyl ether, ii) water, and iii) the extracted morphinan and/or benzylisoquinoline alkaloid(s) (optionally, dissolved in a mixture of the liquefied, sub-critical fluid or super-critical dimethyl ether, and water). One skilled in the art will recognize that in addition to the morphinan and/or benzylisoquinoline alkaloid(s), the mixture of the liquefied, sub-critical fluid or super-critical fluid (such as dimethyl ether) and water further contains extracted non-alkaloid materials including, but not limited to lipids, waxes, tars, peptides, other plant metabolites, etc.

The total extract is then extracted with a suitably selected first aqueous base such as sodium hydroxide, potassium hydroxide and the like, optionally, potassium hydroxide, to extract the phenolic alkaloids (e.g. morphine and oripavine) into the aqueous phase; or extracted with a suitably selected buffer such as mono-potassium phosphate, buffers in a pH in the range of pH 6.0 to pH 7.0, and the like, to extract codeine into the aqueous phase; to yield a biphasic mixture, optionally, a biphasic mixture which is substantially free of emulsion; wherein the aqueous phase is enriched in phenolic alkaloids or codeine, and wherein the organic, pressurized solvent phase is enriched in the non-phenolic alkaloids (e.g. thebaine, noscapine, papaverine, etc.) and further contains non-alkaloid materials.

The organic pressurized phase containing the non-phenolic alkaloids (e.g. thebaine, noscapine, papaverine, etc.) is then further optionally extracted with an aqueous acid; to yield a biphasic mixture, optionally, a biphasic mixture which is substantially free of emulsion; wherein the aqueous phase contains the non-phenolic alkaloids and wherein the organic, pressurized solvent phase contains the non-alkaloid materials such as lipids, waxes, tars, peptides, other plant metabolites, etc. The phases of the biphasic mixture are then separated and the non-phenolic alkaloids isolated according to known methods, for example by adjusting the pH of the aqueous phase a pH in the range of from about pH 7.5 to about pH 11.5 (optionally to a pH in the range of from about pH 8.5 to about pH 10.0, optionally, to a pH in the range of from about 8.5 to about 9.5), to precipitate the non-phenolic alkaloid(s), which non-phenolic alkaloid(s), are then isolated according to known methods, for example, by filtration. The aqueous acid can be an aqueous inorganic acid, an aqueous organic acid or a mixture thereof. Suitable aqueous inorganic acids include, but are not limited to, phosphoric acid, hydrochloric acid, sulfuric acid, or mixtures thereof, optionally, phosphoric acid. Suitable aqueous organic acids include, but are not limited to, lactic acid, formic acid, acetic acid, citric acid, oxalic acid, uric acid or mixtures thereof, or optionally citric acid, oxalic acid or mixtures thereof. Optionally, the aqueous acid is insoluble or difficultly soluble in dimethyl ether.

One skilled in the art will recognize that different morphinan and/or benzylisoquinoline alkaloids may be individually extracted from the total extract, through a series of aqueous base or buffer extractions, wherein the pH of each individual aqueous base or buffer is selected to selectively extract a specific, individual or group of morphinan and/or benzylisoquinoline alkaloid(s). For example, codeine may be optionally extracted by using an aqueous base or buffer with a pH in the range of from about 6.0 to about 7.0 (for example monopotassium phosphate buffer); thebaine may be optionally extracted with an aqueous acid or buffer with a pH less than about pH 4.0, optionally with a pH in the range of from about 2.5 to about 4.0 (for example, phosphoric acid); and morphine and oripavine may be optionally extracted with an aqueous base or buffer with a pH of greater than about 12 (for example sodium hydroxide or potassium hydroxide).

Alternatively, the total extract is extracted with a suitably selected aqueous acid; wherein the pH of the suitably selected aqueous acid is optionally less than about pH 4; to yield a biphasic mixture, optionally a biphasic mixture which is substantially free of emulsion, wherein the aqueous phase contains the morphinan and/or benzylisoquinoline alkaloid(s) and wherein the organic phase contains the non-alkaloid materials such as lipids, waxes, tars, peptides, other plant metabolites, etc. The aqueous phase containing the morphinan and/or benzylisoquinoline alkaloid(s) is then pH adjusted to a pH in the range of from about pH 7.5 to about pH 11.5 (optionally to a pH in the range of from about pH 8.5 to about pH 10.0, optionally to a pH in the range of from about 8.5 to about 9.5), to precipitate the morphinan and/or benzylisoquinoline alkaloid(s), which are then isolated according to known methods, for example, by filtration. The aqueous acid can be an aqueous inorganic acid, an aqueous organic acid or a mixture thereof. Suitable aqueous inorganic acids include, but are not limited to, phosphoric acid, hydrochloric acid, sulfuric acid, or mixtures thereof, optionally, phosphoric acid. Suitable aqueous organic acids include, but are not limited to, lactic acid, formic acid, acetic acid, citric acid, oxalic acid, uric acid or mixtures thereof, or optionally, citric acid, oxalic acid or mixtures thereof. Optionally, the aqueous acid is insoluble or difficultly soluble in dimethyl ether. One skilled in the art will recognize that the organic, pressurized solvent phase may be vented to atmosphere to evaporate the solvent and yield the extracted non-alkaloid materials such as lipids, waxes, tars, peptides, other plant metabolites, etc. as a residue.

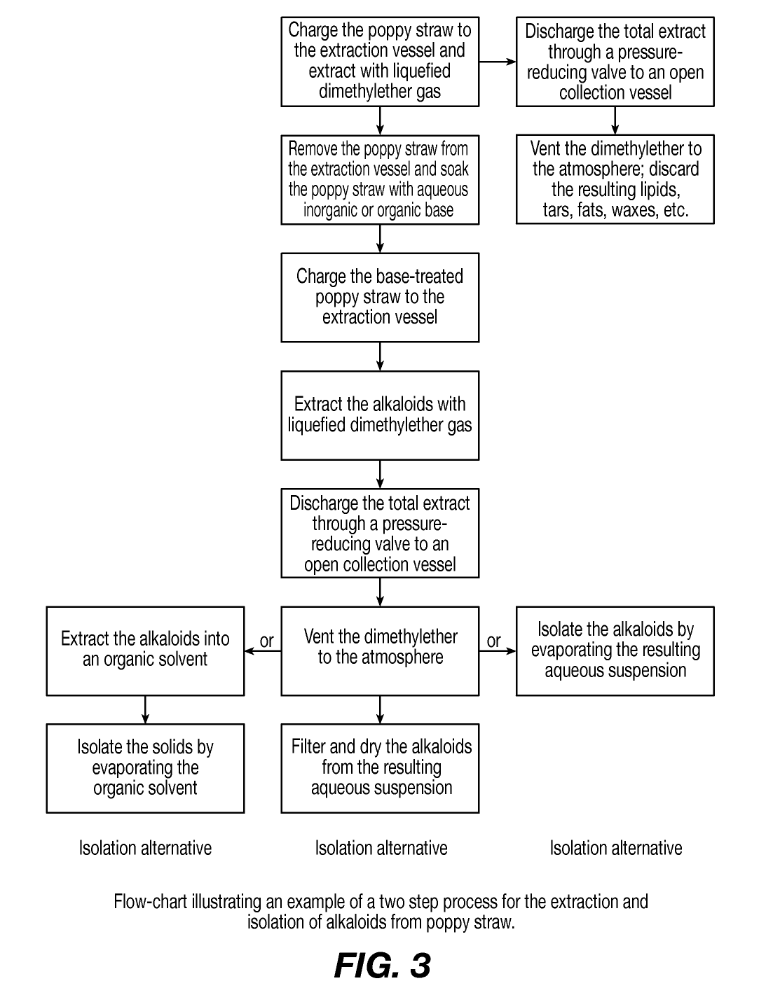

In another embodiment, the present invention is directed to a third extraction method, as illustrated in FIG. 3, comprising charging an extraction vessel with poppy plant containing non-alkaloids and alkaloids (i.e., morphinan and/or benzylisoquinoline alkaloid(s), including phenolic and non-phenolic alkaloids) (for example, poppy straw as exemplified in FIG. 3), which poppy plant has not been treated with a suitably selected base to liberate the alkaloid(s) from the cellular matrix, and contacting said poppy plant with at least one liquefied gas, sub-critical fluid or super-critical fluid as described herein as a first liquefied gas, sub-critical fluid or super-critical fluid to yield an alkaloid containing non-alkaloid material depleted poppy plant and an extract comprising i) the first liquefied gas, sub-critical fluid or super-critical fluid, ii) water, and iii) extracted non-alkaloid materials such as lipids, tars, waxes, peptides, other plant metabolites, etc. In certain embodiments, the extract will not contain significant amounts of the alkaloid(s) other than noscapine. Optionally, the extract contains less than about 25% by weight, optionally, less than about 10%, optionally, less than about 5%, optionally, less than about 2%, of the total available alkaloids, excluding noscapine. The extract comprising the first liquefied gas, sub-critical fluid or super-critical fluid, water, and extracted non-alkaloid materials is then discharged from the vessel.

The alkaloid containing non-alkaloid material depleted poppy plant is then treated (e.g. soaked) with a suitably selected organic base (for example, triethylamine) or inorganic base, such as potassium hydroxide, sodium hydroxide, calcium hydroxide, ammonia, potassium carbonate, sodium carbonate, sodium bicarbonate and the like or mixtures thereof, optionally KOH or NaOH, optionally an aqueous inorganic base, optionally, aqueous calcium hydroxide, optionally, a slurry of 2% w/w aqueous calcium hydroxide; to yield an aqueous mixture containing base treated alkaloid containing, non-alkaloid material depleted poppy plant. One skilled in the art will recognize that treating the alkaloid containing non-alkaloid material depleted poppy plant with the suitably selected organic base or inorganic base liberates the alkaloid(s) from the cellular matrix of the poppy.

The aqueous mixture containing the base treated alkaloid containing, non-alkaloid material depleted poppy plant is then contacted with at least one liquefied gas, sub-critical fluid or super-critical fluid as described herein as a second liquefied gas, sub-critical fluid or super-critical fluid, provided that at least one of, optionally both, the first and/or second liquefied gases, sub-critical fluids or super-critical fluids is a liquefied, super-critical, or sub-critical dimethyl ether, to yield a total extract comprising i) the second liquefied gas, sub-critical fluid or super-critical fluid, ii) water and ii) extracted alkaloid(s) (from the base treated alkaloid containing, non-alkaloid material depleted poppy plant) dissolved in a mixture of second liquefied gas, sub-critical fluid or super-critical fluid and water. The alkaloid(s) are then further isolated as a residue, optionally a solid, according to known methods. In an example, the total extract is vented to release the second liquefied gas, sub-critical fluid or super-critical fluid, and yield an aqueous suspension of the extracted alkaloid(s). The morphinan and/or benzylisoquinoline alkaloid(s)) are then isolated according to known methods, for example by filtration or evaporation of the aqueous phase. In another example, the total extract is extracted with a suitably selected organic solvent such as toluene, n-butanol, tetrahydrofuran or 2-methyl-tetrahydrofuran, and the like or mixtures thereof, optionally toluene; to yield a biphasic mixture, optionally a biphasic mixture which is, optionally, substantially free of emulsion, wherein the extracted morphinan and/or benzylisoquinoline alkaloids are present in the organic layer. The organic and aqueous phases are then separated; and the morphinan and/or benzylisoquinoline alkaloid(s) isolated from the organic phase according to known methods, for example by crystallization or evaporation of the organic solvent.

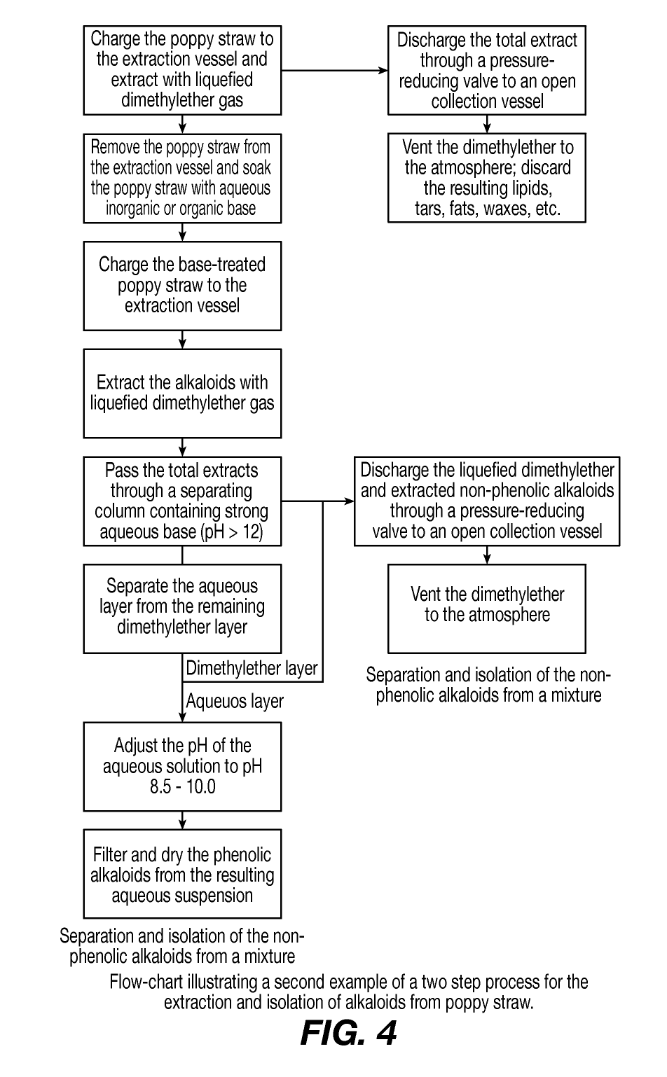

In another embodiment, the present invention is directed to fourth extraction method, as illustrated in FIG. 4, comprising charging an extraction vessel with a poppy plant containing non-alkaloids and alkaloids (i.e., morphinan and/or benzylisoquinoline alkaloid(s), including phenolic and non-phenolic alkaloids) (for example, poppy straw as exemplified in FIG. 4), which poppy plant has not been treated with a suitably selected base to liberate the alkaloid(s) from the cellular matrix, and contacting said poppy plant with at least one liquefied gas, sub-critical fluid or super-critical fluid as described herein as a first liquefied gas, sub-critical fluid or super-critical fluid to yield an alkaloid containing non-alkaloid material depleted poppy plant and an extract comprising i) the first liquefied gas, sub-critical fluid or super-critical fluid, ii) water, and iii) extracted non-alkaloid materials such as lipids, tars, waxes, peptides, other plant metabolites, etc. In certain embodiments, the extract will not contain significant amounts of the alkaloid(s) other than noscapine. Optionally, the extract contains less than about 25% by weight, optionally, less than about 10%, optionally, less than about 5%, or optionally less than about 2%, of the total available alkaloids, excluding noscapine. The extract comprising i) the first liquefied gas, sub-critical fluid or super-critical fluid, ii) water, and iii) the extracted non-alkaloid materials is then discharged from the vessel.

The alkaloid containing non-alkaloid material depleted poppy plant is then treated (e.g. soaked) with a suitably selected organic base (for example, triethylamine) or inorganic base, potassium hydroxide, sodium hydroxide, such as calcium hydroxide, ammonia, potassium carbonate, sodium carbonate, sodium bicarbonate and the like or mixtures thereof, optionally an aqueous inorganic base, optionally, aqueous calcium hydroxide, or optionally, a slurry of 2% w/w aqueous calcium hydroxide; to yield a base treated alkaloid containing, non-alkaloid material depleted poppy plant. One skilled in the art will recognize that treating the alkaloid containing non-alkaloid material depleted poppy plant with the suitably selected organic base or inorganic base liberates the alkaloid(s) from the cellular matrix of the poppy.

The aqueous mixture containing the base treated, alkalodi containing, non-alkaloid material depleted poppy plant is then contacted with at least one liquefied gas, sub-critical fluid or super-critical fluid as described herein as a second liquefied gas, sub-critical fluid or super-critical fluid, provided that at least one of, optionally both, the first and/or second liquefied gases, sub-critical fluids or super-critical fluids is a liquefied, super-critical, or sub-critical dimethyl ether, to yield a total extract comprising i) the second liquefied gas, sub-critical fluid or super-critical fluid, ii) water and iii) extracted alkaloid(s) (from the base treated alkaloid containing, non-alkaloid depleted poppy plant) dissolved in a mixture of the second liquefied gas, sub-critical fluid or super-critical fluid and water.

The total extract is then extracted with a suitably selected second aqueous base of pH greater than 12 (or about 12) such as potassium hydroxide, sodium hydroxide, and the like or mixtures thereof, optionally, potassium hydroxide, to extract the corresponding phenolic alkaloids (e.g. morphine and oripavine) into the aqueous phase (i.e. to yield a biphasic mixture wherein the aqueous phase comprises the phenolic alkaloids and wherein the pressurized organic phase comprises nonphenolic alkaloids); or extracted with a suitably selected buffer such as mono-potassium phosphate, which buffers in a pH in the range of about pH 6.0 to about pH 7.0, and the like, optionally, mono-potassium phosphate; to yield a biphasic mixture, optionally, a biphasic mixture which is substantially free of emulsion; wherein the aqueous phase is enriched in codeine, and wherein the organic phase, pressurized solvent phase (i.e., the second liquefied gas, sub-critical fluid or super-critical fluid) is enriched in non-phenolic alkaloids (e.g. thebaine, noscapine, papaverine, etc.). The biphasic mixture is then separated and the phenolic alkaloids isolated according to known methods, for example by adjusting the pH of the aqueous phase to a pH in the range of from about pH 7.5 to about pH 11.5 (optionally, to a pH in the range of from about pH 8.5 to about pH 10.0, optionally, to a pH in the range of from about 8.5 to about 9.5); to yield a precipitate of the phenolic alkaloid(s), which are then isolated according to known methods, for example, by filtration.

The organic pressurized solvent phase containing the non-phenolic alkaloids (e.g. thebaine, noscapine, papaverine, etc.) is then further optionally extracted with an aqueous acid; to yield a biphasic mixture, optionally, a biphasic mixture which is substantially free of emulsion; wherein the resulting aqueous phase contains the non-phenolic alkaloids. The biphasic mixture is then separated and the non-phenolic alkaloids isolated according to known methods, for example by adjusting the pH of the aqueous phase to a pH in the range of from about pH 7.5 to about 11.5 (optionally, to a pH in the range of from about pH 8.5 to about pH 10.0, optionally, to a pH in the range of from about 8.5 to about 9.5), to precipitate the non-phenolic alkaloid(s), which are then isolated according to known methods, for example, by filtration. The aqueous acid can be an aqueous inorganic acid, an aqueous organic acid or a mixture thereof. Suitable aqueous inorganic acids include, but are not limited to, phosphoric acid, hydrochloric acid, sulfuric acid, or mixtures thereof, optionally, phosphoric acid. Suitable aqueous organic acids include, but are not limited to, lactic acid, formic acid, acetic acid, citric acid, oxalic acid, uric acid or mixtures thereof, or optionally citric acid, oxalic acid or mixtures thereof. Optionally, the aqueous acid is insoluble or difficultly soluble in dimethyl ether.

One skilled in the art will recognize that different morphinan and/or benzylisoquinoline alkaloids may be individually extracted from the total extract, through a series of aqueous base or buffer extractions, wherein the pH of each individual aqueous base or buffer is selected to selectively extract a specific, individual or group of morphinan and/or benzylisoquinoline alkaloid(s). For example, codeine may be optionally, extracted by using an aqueous base or buffer with a pH in the range of from about 6.0 to about 7.0 (for example monopotassium phosphate buffer); thebaine may be optionally, extracted with an aqueous acid or buffer with a pH in less than about pH 4.0, optionally, with a pH in the range of about 2.5 to 4.0 (for example, phosphoric acid); and morphine and oripavine may be optionally, extracted with an aqueous base or buffer with a pH of greater than about 12 (for example sodium hydroxide or potassium hydroxide).

Alternatively, the total extract is extracted with a suitably selected aqueous acid; wherein the pH of the suitably selected aqueous acid (whether organic or inorganic) is optionally, less than about pH 4; to yield a biphasic mixture, optionally, a biphasic mixture which is substantially free of emulsion, wherein the aqueous phase contains the morphinan and/or benzylisoquinoline alkaloid(s). The aqueous phase containing the morphinan and/or benzylisoquinoline alkaloid(s) is then pH adjusted to a pH in the range of from about pH 7.5 to about pH 11.5 (optionally to a pH in the range of from about 8.5 to about pH 10.0, optionally, to a pH in the range of from about 8.5 to about 9.5), to precipitate the morphinan and/or benzylisoquinoline alkaloid(s), which are then isolated according to known methods, for example, by filtration. The aqueous acid can be an aqueous inorganic acid, an aqueous organic acid or a mixture thereof. Suitable aqueous organic acids include, but are not limited to, lactic acid, formic acid, acetic acid, citric acid, oxalic acid, uric acid or mixtures thereof, or optionally citric acid, oxalic acid or mixtures thereof. Suitable aqueous inorganic acids include, but are not limited to, phosphoric acid, hydrochloric acid, sulfuric acid, or mixtures thereof, or optionally, phosphoric acid. Optionally, the aqueous acid is insoluble or difficultly soluble in dimethyl ether.

One skilled in the art will recognize that in the extraction processes of the present invention, specific and/or optional conditions of pressure and temperature may be selected and/or optimized for the particular morphinan and/or benzylisoquinoline alkaloid being extracted, and/or based upon the specific nature of the morphinan and/or benzylisoquinoline alkaloid containing poppy plant. In an embodiment, in the extraction processes of the present invention, the temperature of the dimethyl ether is in the range of from about 0° C. to about 100° C., or any temperature or range of temperatures therein, optionally, in the range of from about 30° C. to about 70° C., optionally, in the range of from about 45° C. to about 55° C.; and the pressure of the dimethyl ether is in the range of from about 4 atm to about 35 atm (about 58 psi to about 515 psi), or any pressure or range of pressures therein, optionally, in the range of from about 10 atm to about 25 atm (about 146 psi to about 367 psi). In another embodiment, in the extraction processes of the present invention, the temperature of the dimethyl ether is about 25° C. and the pressure of the dimethyl ether is about 13.5 atm (about 200 psi).

As a particular advantage, in the extraction processes of the present invention, the contacting of base treated poppy plant (for example, base treated poppy straw) with liquefied, sub-critical or super-critical dimethyl ether also results in the extraction of the water introduced into the poppy straw with the aqueous base treatment. The resulting liquid phase (comprising the liquefied, sub-critical or super-critical dimethyl ether, extracted water, extracted alkaloids and/or extracted non-alkaloid materials) is then separated from the solid, spent poppy plant (for example, poppy straw) such that the spent poppy plant (for example, spent poppy straw) is dry (or dry enough) so as to be suitable for use immediately or directly as a fuel (i.e. without further drying). As used herein, the term “dry” or “dry enough” means less than about 40%, optionally, less than about 30%, optionally, less than about 20%, optionally, less than about 10% water, by weight.

One skilled in the art will further recognize that in the processes of the present invention, once the morphinan and/or benzylisoquinoline alkaloids are extracted from the poppy plant into a liquefied, sub-critical or super-critical dimethyl ether (optionally, liquefied dimethyl ether or, optionally, liquefied, sub-critical dimethyl ether), said morphinan and/or benzylisoquinoline alkaloid(s) may be isolated individually or as any mixture thereof, according to any of the methods as described herein. One skilled in the art will further recognize that in the processes of the present invention, the morphinan and/or benzylisoquinoline alkaloids are further easily isolated from non-alkaloid materials present in the poppy plant (for example poppy straw) and/or present in the dimethyl ether extract.

In an embodiment of the present invention, phenolic alkaloids (such as morphine, oripavine, and the like) are separated from the non-phenolic alkaloids (such as codeine, thebaine, and the like) and optionally, from non-alkaloid materials (such as fats, tars, waxes, peptides, other plant metabolites, and the like) by extraction of a total extract with a suitably selected aqueous solution of pH greater than about pH 12, for example an aqueous potassium hydroxide solution. One skilled in the art will recognize that in said extraction, the phenolic alkaloids partition into the aqueous layer, whereas the non-phenolic alkaloids remain in the organic layer.

In another embodiment of the present invention, the morphinan and/or benzylisoquinoline alkaloids (which are polar compounds) are separated from non-alkaloid materials (such as tars, fats, waxes, lipids, peptides, other plant metabolites, and the like) by extraction of a total extract with a suitably selected aqueous solution of pH less than about pH 4, for example an aqueous phosphoric acid solution. One skilled in the art will recognize that in said extraction, the polar morphinan and/or benzylisoquinoline alkaloid(s) partition into the aqueous layer, whereas the non-alkaloid materials remain in the organic layer.

Adjustments in pH as referenced anywhere herein may be accomplished using any suitable, optionally pharmaceutically acceptable, pH adjusting agent, including bases, such as sodium hydroxide or acids such as hydrochloric acid.

PURITY AND YIELD In an embodiment of the present invention, a morphinan or benzylisoquinoline alkaloid is isolated from poppy plants at a purity in the range of from about 25% assay to about 95% assay by weight, or any amount or range therein, optionally, at a purity in the range of from about 50% to about 95% assay, optionally at a purity in the range of from about 80% to about 95% assay as determined using the UPLC Analysis method described in the Examples section; and/or at a yield in the range of from about 25% to about 100% (relative to total available morphinan or benzylisoquinoline alkaloid) by weight, or any amount or range therein, optionally at a yield in the range of from about 50% to about 100%, optionally at a yield in the range of about 85% to about 100%.

In another embodiment of the present invention, thebaine is isolated from a suitably selected poppy plant at a purity in the range of from about 24% assay to about 95% assay by weight, or any amount or range therein, optionally at a purity in the range of from about 50% to about 95% assay, optionally at a purity in the range of from about 80% to about 95% assay; and/or at a yield in the range of from about 50% to about 100% (relative to total available thebaine) by weight, or any amount or range therein, optionally, at a yield in the range of from about 75% to about 100%, optionally at a yield in the range of about 85% to about 100%.

In another embodiment of the present invention, oripavine is isolated from a suitably selected poppy plant at a purity in the range of from about 24% assay to about 95% assay by weight, or any amount or range therein, optionally, at a purity in the range of from about 50% to about 95% assay, optionally, at a purity in the range of from about 80% to about 95% assay; as determined using the UPLC Analysis method described in the Examples section and/or at a yield in the range of from about 32% to about 100% (relative to total available oripavine) by weight, or any amount or range therein, assay at a yield in the range of from about 75% to about 100%, optionally, at a yield in the range of about 85% to about 100%.

In another embodiment of the present invention, morphine is isolated from a suitably selected poppy plant at a purity in the range of from about 29% assay to about 95% assay by weight, or any amount or range therein, optionally at a purity in the range of from about 50% to about 95% assay, optionally at a purity in the range of from about 80% to about 95% assay as determined using the UPLC Analysis method described in the Examples section; and/or at a yield in the range of from about 44% to about 100% (relative to total available morphine) by weight, or any amount or range therein, optionally, at a yield in the range of from about 75% to about 100%, optionally, at a yield in the range of about 85% to about 100%.

In another embodiment of the present invention, codeine is isolated from a suitably selected poppy plant at a purity in the range of from about 20% assay to about 99% assay by weight, or any amount or range therein, optionally, at a purity in the range of from about 70% to about 98% assay, optionally, at a purity in the range of from about 80% to about 98% assay as determined using the UPLC Analysis method described in the Examples section; and/or at a yield in the range of from about 20% to about 99% (relative to total available codeine) by weight, or any amount or range therein, optionally, at a yield in the range of from about 60% to about 99%, optionally, at a yield in the range of about 80% to about 99%.

In another embodiment of the present invention, noscapine is isolated from a suitably selected poppy plant at a purity in the range of from about 20% to about 99% assay by weight, or any amount or range therein, optionally, at a purity in the range of from about 70% to about 98% assay, optionally, at a purity in the range of from about 80% to about 98% assay; and/or at a yield in the range of from about 20% to about 99% (relative to total available noscapine) by weight, or any amount or range therein, optionally, at a yield in the range of from about 60% to about 99%, optionally, at a yield in the range of about 80% to about 99%.

One skilled in the art will recognize that any of the process steps as herein described may be run under laboratory, large scale and/or manufacturing conditions, under batch or continuous process conditions. Optionally, any one or mixture of one or more of the morphinan and/or benzylisoquinoline alkaloids are isolated, according to any of the methods as herein described, or according to any method as would be known to those skilled in the art, via continuous process conditions. One skilled in the art will further recognize that any one or more individually selected extractions may be optionally run in a countercurrent mode. One skilled in the art will recognize that wherein the processes (or individual process steps) of the present invention as described herein, the use of dimethyl ether results in a biphasic mixture which is, optionally, substantially free of emulsion, said processes (or individual process steps) are particularly suited for running under continuous process conditions. (One skilled in the art will further recognize that the formation of an emulsion is not desirable when running under continuous process conditions, as the emulsion will diminish the efficiency of the separations).

### EXAMPLES

The following Examples are set forth to aid in the understanding of the invention, and are not intended and should not be construed to limit in any way the invention set forth in the claims which follow thereafter.

In the Examples which follow herein, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Laboratory-Scale Extraction Apparatus and Example

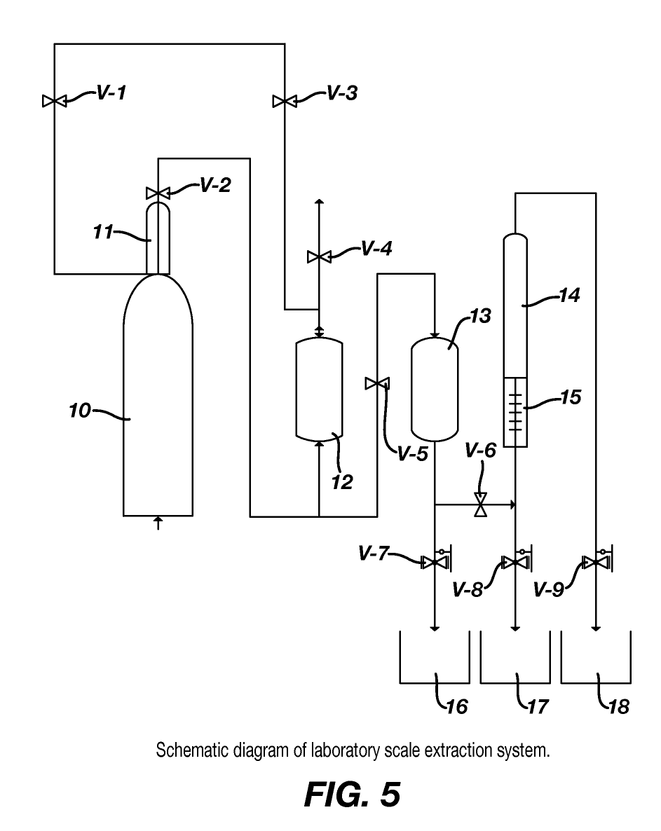

Described below is a representative example of the procedure used in the extraction processes of Examples 1-15, with reference to the specific extraction apparatus components, as shown in FIG. 5.

Morphinan alkaloid containing poppy straw was initially charged to the extraction vessel (13). Dimethyl ether, as a liquefied gas was compressed by nitrogen (controlled by needle valve (V-1)) to the desired pressure. The compressed dimethyl ether gas was discharged from the cylinder (10) through a dip tube (11) and metered through a needle valve (V-2). Compressed dimethyl ether gas was delivered at a flow rate in the range of from about 20 g/min to about 200 g/min (controlled by a needle valve (V-3)) to a surge tank/pre-heater (12) and then via a needle valve (V-5) to the extraction vessel (13), which was arranged in series with said surge tank/pre-heated. Pressure was controlled to within ±5 bar and the flow rate was controlled by a pressure-reduction valve (V-4). The temperature of both the surge tank/pre-heater (12) and extraction vessel (13) was maintained to within 2° C., as measured by thermocouples (not shown) located on the outside skin of both vessels and controlled by electrical heating tape that was regulated by a PID temperature controller (Omega Engineering) (not shown). Downstream of the extraction vessel (13), the dimethyl ether/water mixture laden with dissolved morphinan alkaloid(s) from the charge (i.e. the total extract), was expanded to atmospheric pressure via the pressure reduction valve (V-7) causing the extracted morphinan alkaloids to precipitate. The extracted precipitate was collected in a pre-weighed collection vessel (16).

In some experiments (as noted in the details provided for the individual Examples below), the morpinan alkaloid(s) were further purified or separated into phenolic-alkaloids and non-phenolic alkaloids by adding an aqueous solution of appropriate pH to a phase separation column (14) and passing the dimethyl ether/water mixture laden with dissolved morphinan alkaloids (i.e. the total extract) directly from the extraction vessel (13) through a needle valve (V-6) to the bottom of the phase separation column (14) (directional movement into phase separation column (14) not shown). The phase separation column (14) was, optionally, equipped with a four inch length of stainless steel distillation column packing (15) inserted in the base of the column as noted above to function as a static mixer, to improve partitioning of the morphinan alkaloids from the dimethyl ether/water mixture into the aqueous extraction solution. The dimethyl ether layer exiting via the top of the phase separation column (14), and was expanded to atmospheric pressure via the pressure reduction valve (V-9) causing the extracted morphinan alkaloid(s) to precipitate and collect as an aqueous suspension in a pre-weighed collection vessel (18). On completion of the phase separation, the aqueous phase was drained through pressure-reduction valve (V-8) to a pre-weighed collection vessel (17). The extracted, separated morphinan alkaloids collected in the collection vessels (17 and 18), present as a suspension in the aqueous solution, were isolated by for example, filtration. Any remaining dimethyl ether layer was drained through pressure-reduction valve (V-8) to a second pre-weighed collection vessel (not shown) combined with any dimethyl ether layer drained from pressure reduction valve (V-9).

All process vessels, tubing and valves were constructed of either 304 or 316 stainless steel with ¼″ SWAGELOC fittings. The phase separation vessel was constructed of glass to allow for visual inspection of phase separations, and protected by a perforated stainless steel sleeve. Specific components used in the laboratory apparatus were as follows:
PID Temperature control: J-type skin thermocouple (Omega Engineering) P/N SA1-J-SC); Temperature Controller (Omega Engineering) Model #: CN76000
Extractor: 500 ml stainless steel (Thar Technologies), inside dimensions 5cm ID×20cm length
Glass Column: 1″ ID×15″ long glass column (Chem-Flowtronics) P/N: SF-1150-SS-25-HW-PS
Back Pressure Regulator: (Tescom) Model #: 26-2321-28-302
Pressure gauges: (Ashcroft Instruments) 20W1005PH 02L 600#
Valves: SWAGLOC needle valves for ¼″ tubing, P/N: SS-1 VS4
Static Mixer: a four inch length of stainless steel distillation column packing inserted in the base of the column functioned as a static mixer. “No packing” when used with respect to a phase separation column means that the phase separation column has no static mixer associated with it.

In the Examples which follow herein, reference may be made to specific extraction components used in the experiments described therein. The complete extraction system used in said Examples, was as shown schematically in FIG. 5. The assay (wt %) for extracted/isolated alkaloids as indicated in each of Examples 1-13 was obtained using the following UPLC Analysis Method.

### UPLC Analysis Method

### Sample Preparation Solution:

20 mL of concentrated formic acid and 100 mL methanol were added to 1880 mL of DI (deionized) water and the resulting solution mixed well. Using an analytical balance, the amount of sample was weighed into a clean disposable snap-seal container. Using a dispenser or similar measuring apparatus the volume of 1% formic acid/5% methanol sample diluent was added to the container. A small stir bar was placed in the container, the solution was capped, and stirred for at least 20 minutes. After mixing, a portion of the solution was immediately filtered through a 0.2 μm Pall Life Sciences Acrodisc CR PTFE membrane syringe filter or equivalent into an UPLC vial prior to analysis.

### Equipment:

Waters Acquity™ UPLC Separation Module, Analytical Balance, Sonicator Bath

### Reagents:

Mobile Phase A1 (10 mM Mixed Phosphate Buffer in 95:5 Water/Methanol)

Mobile Phase B1 (100% pre-filtered HPLC Grade or better Methanol)

Mobile Phase A2 (1% Formic Acid)-Column Flushing/Storage Only

Mobile Phase B2 (100% pre-filtered HPLC Grade Methanol)-Column Flushing/Storage Only:

Strong Needle Wash (80:20:1% Methanol/Water/Formic Acid)

Weak Needle Wash (10:90:1% Methanol/Water/Formic Acid)

### Chromatographic Instrument Setup:

| Column: | Waters Acquity ™ BEH C18, 50 × 2.1 mm, | |
|---|---|---|
| | 1.7 μm (Part No. 186002350) | |
| Column Pre-Filter: | 0.2 μm column pre-filter attached (Part No. | |
| | 289002078) | |
| Detector Inlet Tubing: | 0.004 ID inlet detector tubing (Part No. | |
| | 430001748) | |
| Data Mode: | Absorbance with MBF | |
| Auto Zero Function: | Maintain Baseline | |
| Injection Volume | 0.5 | μL |
| Column Temp: | 55° | C. |
| Sample Comp. Temp: | 20° | C. |
| Wavelength: | 284 | nm |
| Flow Rate: | 0.70 | mL/min. |
| Sampling Rate: | 40 | pt./sec. |
| Filtering Constant: | 0.05 | sec. |
| Sample Loop Size: | 2 or 5 | μL |
| Run Time: | 5 | minutes |

### Gradient Timetable:

| Time (min.) | % A1 | % B1 | Curve |
|---|---|---|---|
| Initial | 100.0 | 0.0 | Initial |
| 1.75 | 70.0 | 30.0 | 3 |
| 3.00 | 55.0 | 45.0 | 6 |
| 4.25 | 100.0 | 0.0 | 9 |
| 5.00 | 100.0 | 0.0 | 1 |

The samples were automatically injected (injection volume 0.5 μL) and chromatographed by the Acquity™ UPLC along with standard reference alkaloids. After the sample set had been run by the Acquity™ UPLC, the peaks were identified by comparison with the standard reference alkaloids that were run in the sample set. Typical retention times were as follows:

| | | Approximate |
|---|---|---|
| Component | Abbreviation | Retention Time |
| Morphine | AMA | 1.2 minutes |
| Oripavine | AOA | 1.8 minutes |
| Codeine | ACA | 2.4 minutes |
| Thebaine | ATA | 3.2 minutes |

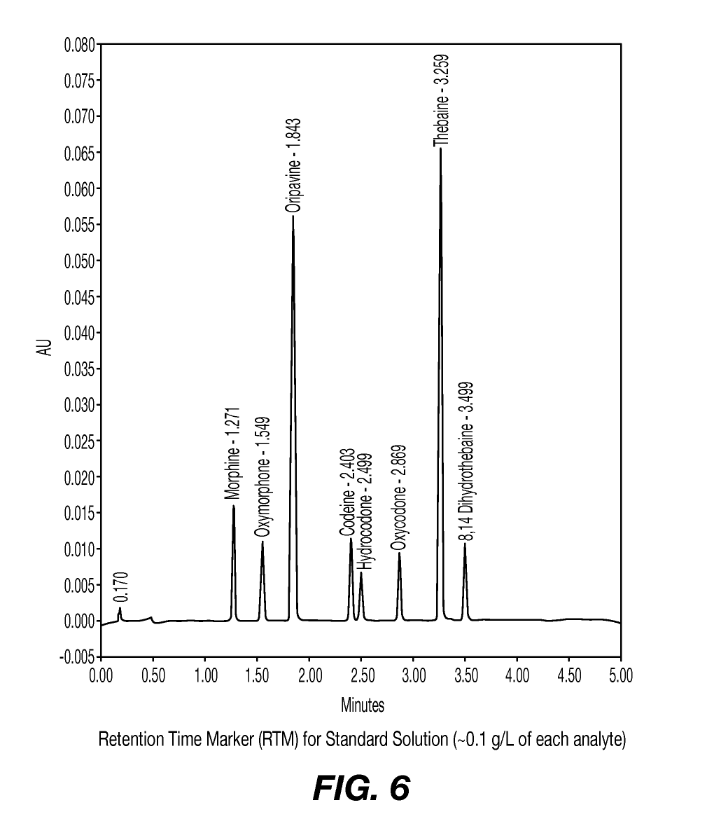

The separations obtained using this method were as shown in FIG. 6. Empower 2 chromatography software (Waters Corporation, Milford, Mass.) was used to identify peaks, calculate peak areas, and quantitate the assays of the components on a % w/w basis. The data was then exported to an Excel spreadsheet.

### Example 1

### One-Step Extraction of Thebaine From a Thebaine-Rich Poppy Straw

A slurry of 1.1 grams of calcium hydroxide in 52.7 grams of water was prepared and mixed with 49.7 grams of a milled, thebaine-rich poppy straw from a thebaine-rich mutant of a Papaver somniferum poppy. The poppy straw thus treated was charged to the extraction vessel (13) and the chamber of extraction vessel (13) was closed. A solution of 5.4 grams of potassium hydroxide in 5.5 grams of water was added to the phase separating column (14). Liquid dimethyl ether (ca. 1.1 L) at 50° C. was passed through the extraction vessel (13) and the resulting extract was passed through the phase separation column (14) (no packing) containing the aqueous potassium hydroxide solution (directional movement into phase separation column (14) not shown). The liquid dimethyl ether layer containing the extracted materials exited through the top of the phase separation column (14) and was passed into open collection vessel (18) through pressure reduction valve (V-9). When the extraction was complete, the aqueous phase (43.6 grams) was separated through pressure reduction valve (V-8) to collection vessel (17). The remaining dimethyl ether extract in the phase separation column (14) was drained through pressure reduction valve (V-8) into collection vessel (17) and combined with the dimethyl ether extract in open collection vessel (18). The dimethyl ether was vented from the open collection vessel (18) to yield a slurry in water weighing 37.6 grams. The water was evaporated on a rotary evaporator at 60° C./40 mbar to yield 2.4 grams of a solid. The solid was then analyzed using the UPLC analysis method described above.

Table 1 below, shows the analytical results for the isolated solid (Wt % thebaine).

TABLE 1
Analytical results for Example 1

| Sample | Weight (grams) | Thebaine Assay (Wt %) |
|---|---|---|
| Poppy straw | 49.7 | 2.9 (1.44 gms) |
| Extract | 2.4 | 62 (1.48 gms) |

Substantially all of the thebaine was recovered from the charge of poppy straw, with a measured yield 103% of theoretical.

### Example 2

### One-Step Extraction of Thebaine From a Thebaine-Rich Poppy Straw On-Line Extraction into Phosphoric Acid Solution

A slurry of 1.15 grams of calcium hydroxide in 50.3 grams of water was prepared and mixed with 49.1 grams of a milled, thebaine-rich poppy straw from a thebaine-rich mutant of a Papaver somniferum poppy. The poppy straw thus treated was charged to the extraction vessel (13) and the chamber of extraction vessel (13) was closed. A solution of 5.4 grams of phosphoric acid (85%) in 15.6 grams of water was added to the phase separating column (14) equipped with the static mixer. Liquid dimethyl ether (ca. 1.1 L) at 50° C. was passed through the extraction vessel (13) and the resulting extract was passed through the phase separation column (14) containing the aqueous phosphoric acid solution (directional movement into phase separation column (14) not shown). The liquid dimethyl ether layer containing the extracted materials exited through the top of the phase separation column (14) and was passed into open collection vessel (18) through pressure reduction valve (V-9). When the extraction was complete, the aqueous phase (32.1 grams) was separated through pressure reduction valve (V-8) to collection vessel (17). The remaining dimethyl ether extract in the phase separation column (14) was drained through pressure reduction valve (V-8) and combined with the dimethyl ether extract in open collection vessel (18). The dimethyl ether was vented from the open collection vessel (18) to yield a slurry in water. The water was evaporated on a rotary evaporator at 60° C./40 mbar to yield 1.1 grams of an oily solid (Extract 1).

The pH of the phosphoric acid extract was adjusted to a pH in the range of pH 9-10 by the addition of 28% aqueous ammonia. The mixture was then extracted with toluene (3×10 mL), the toluene extracts were combined, and the toluene distilled from the extracts on a rotary evaporator at 60° C./40 mbar. The residues were rinsed into a sample vial and the sample was dried overnight in a vacuum oven at 55° C./40 mbar to yield 1.1 grams of a light brown crystalline material (Extract 2). Extracts 1 and 2 were then analyzed using the UPLC analysis method described above. Table 2 below, shows the analytical results for the isolated solid (Wt % thebaine).

TABLE 2
Analytical results for Example 2

| Sample | Weight (grams) | Thebaine Assay (Wt %) |
|---|---|---|
| Poppy straw | 49.1 | 2.9 (1.42 gms) |
| Extract 1 | 3.9 | 0.2 (0.008 gms) |
| Extract 2 | 1.1 | 84.9 (0.94 gms) |

The yield was 66% of theoretical.

### Example 3

### Two Step Extraction of Thebaine From a Thebaine-Rich Mutant Poppy

Milled, thebaine-rich poppy straw (50.0 grams) from a thebaine-rich mutant of a Papaver somniferum poppy (TED) was charged to the extraction vessel (13) and the chamber of extraction vessel (13) was closed. Liquid dimethyl ether (ca. 1.1 L) at 25° C. was passed through the extraction vessel (13) and the resulting extract was passed through the pressure reduction valve (V-7) to open collection vessel (16). The dimethyl ether was vented from the open collection vessel (16) to yield an oil, which on further drying under vacuum yielded a tar (0.7 grams).

The extracted poppy straw was removed from the extraction vessel (13) and mixed with a slurry of 1.1 grams of calcium hydroxide in 52.5 grams of water. The poppy straw thus treated was re-charged to the extraction vessel (13) and the chamber of extraction vessel (13) was closed. A solution of 5.5 grams of potassium hydroxide in 5.2 grams of water was added to the phase separating column (no packing) (14). Liquid dimethyl ether (ca. 1.1 L) at 50° C. was passed through the extraction vessel (13) and the resulting extract was passed through the phase separation column (14) containing the aqueous potassium hydroxide solution (directional movement into phase separation column (14) not shown). The liquid dimethyl ether layer containing the extracted materials exited through the top of the phase separation column (14) and is passed into open collection vessel (18) through pressure reduction valve (V-9). When the extraction was complete, the aqueous phase (37.0 grams) was separated through pressure reduction valve (V-8) to a collection vessel (17) and then evaporated on a rotary evaporator at 60° C./40 mbar to yield 0.7 grams of a residue (Extract 1). The remaining dimethyl ether extract in the phase separation column (14) was drained through pressure reduction valve (V-8) and combined with the dimethyl ether extract in open collection vessel (18). The dimethyl ether was vented from the open collection vessel (18) to yield a slurry in water weighing 44.5 grams. The water was evaporated on a rotary evaporator at 60° C./40 mbar to yield 1.8 grams of a solid (Extract 2). Extracts 1 and 2 were then analyzed using the UPLC analysis method described above. Table 3 below, shows the analytical results for the isolated solid (Wt % thebaine).

TABLE 3
Analytical results for Example 3

| Sample | Weight (grams) | Thebaine Assay (Wt. %) |
|---|---|---|
| Poppy straw | 49.7 | 2.9 (1.44 gms) |
| Extract 1 | 0.7 | 4 (0.003 gms) |
| Extract 2 | 1.8 | 83.3 (1.50 gms) |

Substantially all of the thebaine was recovered from the charge of poppy straw. The yield was 102% of theoretical and the assay of the resulting thebaine is 83%.

### Example 4

### Two-Step Extraction of Thebaine From a thebaine-Rich Poppy Straw Effect of Higher Extraction Temperature (80° C.)

Milled, thebaine-rich poppy straw (50.0 grams) from a thebaine-rich mutant of a Papaver somniferum poppy (TED) was charged to the extraction vessel (13) and the chamber of extraction vessel (13) was closed. Liquid dimethyl ether (ca. 1.1 L) at 50° C. was passed through the extraction vessel (13) and the resulting extract was passed through the pressure reduction valve (V-7) to open collection vessel (16). The dimethyl ether was vented from the open collection vessel (16) to yield an oil, which on further drying under vacuum yielded a tar (Extract 1; 1.1 grams).

The extracted poppy straw was removed from the extraction vessel (13) and mixed with a slurry of 1.0 grams of calcium hydroxide in 51.7 grams of water. The poppy straw thus treated was re-charged to the extraction vessel (13) and the chamber of extraction vessel (13) was closed. Liquid dimethyl ether (ca. 1.1 L) at 80° C. was passed through the extraction vessel (13) and the resulting extract was passed through pressure reduction valve (V-7) to open collection vessel (16). The dimethyl ether was vented from the open collection vessel (16) to yield a slurry in water. The solids were collected by filtration and dried in a vacuum oven at 60° C./40 mbar overnight (Extract 2). Extracts 1 and 2 were then analyzed using the UPLC analysis method described above. Table 4 below, shows the analytical results for the isolated solid (Wt % thebaine).

TABLE 4
Analytical results for Example 4

| Sample | Weight (grams) | Thebaine Assay (Wt. %) |
|---|---|---|
| Poppy straw | 50.1 | 2.9 (1.45 gms) |
| Extract 1 | 1.1 | 21 (0.23 gms) |
| Extract 2 | 1.4 | 70 (0.98 gms) |

Substantially all of the thebaine was recovered from the charge of poppy straw. The overall yield was 91% of theoretical and the assay of the resulting thebaine in the concentrated Extract 2 was 70%.

### Example 5

### Two-Step Extraction of Thebaine From a Thebaine-Rich Poppy Straw Effect of Higher Extraction Temperature (100° C.)

Milled, thebaine-rich poppy straw (48.9 grams) from a thebaine-rich mutant of a Papaver somniferum poppy (TED) was charged to the extraction vessel (13) and the chamber of extraction vessel (13) was closed. Liquid dimethyl ether (ca. 1.1 L) at 50° C. was passed through the extraction vessel (13) and the resulting extract was passed through pressure reduction valve (V-7) to open collection vessel (16). The dimethyl ether was vented from the open collection vessel (16) at ambient conditions to yield an oil, which on further drying under vacuum yielded a tar (1.3 grams).

The extracted poppy straw was removed from the extraction vessel (13) and mixed with a slurry of 1.1 grams of calcium hydroxide in 51.0 grams of water. The poppy straw thus treated was re-charged to the extraction vessel (13) and the chamber of extraction vessel (13) was closed. Liquid dimethyl ether (ca. 1.1 L) at 100° C. was passed through the extraction vessel (13) and the resulting extract was passed through pressure reduction valve (V-7) to open collection vessel (16). The dimethyl ether was vented from the open collection vessel (16) at ambient conditions to yield a slurry in water. The solids were collected by filtration and dried in a vacuum oven at 60° C./40 mbar overnight. Extracts 1 and 2 were then analyzed using the UPLC analysis method described above. Table 5 below, shows the analytical results for the isolated solid (Wt % thebaine).

TABLE 5
Analytical results for Example 5

| Sample | Weight (grams) | Thebaine Assay (Wt. %) |
|---|---|---|
| Poppy straw | 48.9 | 2.9 (1.42 gms) |
| Extract 1 | 1.3 | 10 (0.13 gms) |
| Extract 2 | 1.4 | 73.5 (1.03 gms) |

The majority of the thebaine was recovered from the charge of poppy straw. The overall yield was 83.5% of theoretical and the assay of the resulting thebaine in the concentrated Extract 2 was 73.5%.

### Example 6

### Two-Step Extraction of Thebaine From TED Poppy Straw Use of an Organic Base for Extraction

Milled, thebaine-rich poppy straw (48.8 grams) from a thebaine-rich mutant of a Papaver somniferum poppy (TED) was charged to the extraction vessel (13) and the chamber of extraction vessel (13) was closed. Liquid dimethyl ether (ca. 1.1 L) at 50° C. was passed through the extraction vessel (13) and the resulting extract was passed through pressure reduction valve (V-7) to open collection vessel (16). The dimethyl ether was vented from the open collection vessel (16) to yield an oil, which on further drying under vacuum yielded a tar (0.8 grams).

The extracted poppy straw was removed from the extraction vessel (13) and mixed with a solution of 20.5 grams of triethylamine in 50.1 grams of water. The poppy straw thus treated was re-charged to the extraction vessel (13) and the chamber of extraction vessel (13) was closed. A solution of 5.2 grams of potassium hydroxide in 5.8 grams of water was added to the phase separating column (14). Liquid dimethyl ether (ca. 1.1 L) at 50° C. was passed through the extraction vessel (13) and the resulting extract was passed through the phase separation column (14) containing the aqueous potassium hydroxide solution (directional movement into phase separation column (14) not shown). The liquid dimethyl ether layer containing the extracted materials exited through the top of the phase separation column (14) and was passed into open collection vessel (18) through pressure reduction valve (V-9). When the extraction was complete, the aqueous phase (31.8 grams) was separated through pressure reduction valve (V-8) to collection vessel (17) (Extract 1). The remaining dimethyl ether extract in the phase separation column (14) was drained through pressure reduction valve (V-8) and combined with the dimethyl ether extract in open collection vessel (18). The dimethyl ether gas was vented from the open collection vessel (18) to yield a solution in water/triethylamine weighing 55.5 grams. The water and triethylamine were evaporated on a rotary evaporator at 60° C./40 mbar to yield 1.9 grams of a solid (Extract 2). Extracts 1 and 2 were then analyzed using the UPLC analysis method described above. Table 6 below, shows the analytical results for the isolated solid (Wt % thebaine).

TABLE 6
Analytical results for Example 6

| Sample | Weight (grams) | Thebaine Assay (Wt. %) |
|---|---|---|
| Poppy straw | 49.7 | 2.9 (1.44 gms) |
| Extract 1 | 0.8 | 7.5 (0.06 gms) |
| Extract 2 | 1.9 | 74.6 (1.42 gms) |

Substantially all of the thebaine was recovered from the charge of poppy straw. The yield was 105% of theoretical and the assay of the resulting thebaine is 75%.

### Example 7

### One-Step Extraction of Codeine from a Codeine-Rich Poppy Straw; On-Line Extraction into Mono Potassium Phosphate Buffer Solution

A slurry of 1.15 grams of calcium hydroxide in 50.5 grams of water was prepared and mixed with 48.8 grams of a milled, codeine-rich poppy straw from a codeine-rich mutant of a Papaver somniferum poppy. The poppy straw thus treated was charged to the extraction vessel (13) and the chamber of extraction vessel (13) was closed. A solution of 5.2 grams of potassium monophosphate in 16.5 grams of water was added to the phase separating column (14) equipped with the static mixer. Liquid dimethyl ether (ca. 1.1 L) at 50° C. was passed through the extraction vessel (13) and the resulting extract was passed through the phase separation column (14) containing the aqueous potassium monophosphate solution (directional movement into phase separation column (14) not shown). The liquid dimethyl ether layer containing the extracted materials exited through the top of the phase separation column (14) and was passed into open collection vessel (18) through pressure reduction valve (V-9). When the extraction was complete, the aqueous phase (46.6 grams) was separated through pressure reduction valve (V-8) to collection vessel (17). The remaining dimethyl ether extract in the phase separation column (14) was drained through pressure reduction valve (V-8) and combined with the dimethyl ether extract in open collection vessel (18). The dimethyl ether gas was vented from the open collection vessel (18) to yield a slurry in water. The slurry from open collection vessel (18) was acidified with 10% acetic acid solution (final pH=3.8). The acidified solution was extracted with toluene (3×10 mL), the toluene extracts were combined, and the toluene was distilled on a rotary evaporator at 60° C./40 mbar. The residues were rinsed into a sample vial and the sample was dried overnight in a vacuum oven at 55° C./40 mbar to yield 1.0 grams of a black tarry material (Extract 1).

The pH of the aqueous layer was adjusted to pH 9-10 by the addition of 28% aqueous ammonia. The mixture was then extracted with toluene (3×10 mL), the toluene extracts were combined, and the toluene distilled on a rotary evaporator at 60° C./40 mbar. The residues were rinsed into a sample vial and the sample was dried overnight in a vacuum oven at 55° C./40 mbar to yield 0.4 grams of a brown solid (Extract 2).

The potassium monophosphate aqueous phase (pH 6.0) collected in collection vessel (17) was neutralized by the addition of 28-30% ammonium hydroxide to pH 9.8. The mixture was then extracted with toluene (3×10 mL), the toluene extracts were combined, and the toluene distilled on a rotary evaporator at 60° C./40 mbar. The residues were rinsed into a sample vial and the sample was dried overnight in a vacuum oven at 55° C./40 mbar to yield 0.7 grams of a crystalline off-white solid (Extract 3). Extracts 1 through 3 were then analyzed using the UPLC analysis method described above. Table 7 below, shows the analytical results for the isolated solid (Wt % thebaine and Wt % codeine).

TABLE 7
Analytical results for Example 7

| | Weight | Codeine | Thebaine assay |
|---|---|---|---|
| Sample | (grams) | Assay (Wt. %) | (Wt. %) |
| Poppy straw | 48.8 | 2.29 (1.12 gms) | 0.38 (0.19 gms) |
| Extract 1 (tars etc) | 1.0 | ND | 0.2 (0.002 gms) |
| Extract 2 (alkaloids | 0.4 | 39.8 (0.16 gms) | 23.9 (0.10 gms) |
| from DME residues) | | | |
| Extract 3 (alkaloids | 0.7 | 83.2 (0.58 gms) | 6.7 (0.047 gms) |
| from KH₂PO₄ extract) | | | |

The recovery of codeine was 68% of theoretical. The recovery of thebaine was 78% of theoretical

### Example 8

### Two-Step Extraction of Codeine and Thebaine From a Codeine-Rich Poppy Straw

Milled, codeine-rich poppy straw (50.0 grams) from a codeine-rich mutant of a Papaver somniferum poppy (TASMAN) was charged to the extraction vessel (13) and the chamber of extraction vessel (13) was closed. Liquid dimethyl ether (ca. 1.1 L) at 25° C. was passed through the extraction vessel (13) and the resulting extract was passed through the pressure reduction valve (V-7) to open collection vessel (16). The dimethyl ether was vented from the open collection vessel (16) to yield an oil, which on further drying under vacuum yielded a tar (0.8 grams).

The extracted poppy straw was removed from the extraction vessel (13) and mixed with a slurry of 1.1 grams of calcium hydroxide in 51.0 grams of water. The poppy straw thus treated was re-charged to the extraction vessel and the chamber of extraction vessel (13) was closed. A solution of 5.0 grams of potassium hydroxide in 5.0 grams of water was added to the phase separating column (14). Liquid dimethyl ether (ca. 1.1 L) at 50° C. was passed through the extraction vessel (13) and the resulting extract was passed through the phase separation column (no packing) (14) containing the aqueous potassium hydroxide solution (directional movement into phase separation column (14) not shown). The liquid dimethyl ether layer containing the extracted materials exited through the top of the phase separation column (14) and was passed into open collection vessel (18) through pressure reduction valve (V-9). When the extraction was complete, the aqueous phase (39.3 grams) was separated through pressure reduction valve (V-8) to collection vessel (17). The aqueous phase was then evaporated on a rotary evaporator at 60° C./40 mbar to yield 0.9 grams of a residue (Extract 1). The remaining dimethyl ether extract in the phase separation column (14) was drained through pressure reduction valve (V-8) and combined with the dimethyl ether extract in open collection vessel (18). The dimethyl ether was vented from the open collection vessel to yield a slurry in water weighing 45.1 grams. The water was evaporated on a rotary evaporator at 60° C./40 mbar to yield 1.5 grams of a solid (Extract 2). Extracts 1 and 2 were then analyzed using the UPLC analysis method described above. Table 8 below, shows the analytical results for the isolated solid (Wt % thebaine and Wt % codeine).

TABLE 8
Analytical results for Example 8

| | Weight | Codeine | Thebaine assay |
|---|---|---|---|
| Sample | (grams) | Assay (Wt. %) | (Wt. %) |
| Poppy straw | 50.0 | 2.29 (1.15 gms) | 0.38 (0.19 gms) |
| Extract 1 | 0.8 | 2.1 (0.017 gms) | 0.9 (0.007 gms) |
| Extract 2 | 1.5 | 49.0 (0.74 gms) | 8.0 (0.12 gms) |

The recovery of codeine was 68% of theoretical and the assay of the resulting codeine is 49%.

### Example 9

### One-Step Extraction of Oripavine and Thebaine From a Poppy Straw Rich in Both Thebaine and Oripavine; On-Line Extraction into Potassium Hydroxide Solution

A slurry of 1.18 grams of calcium hydroxide in 51.0 grams of water was prepared and mixed with 49.8 grams of a poppy straw containing both thebaine (a non-phenolic alkaloid) and oripavine (a phenolic alkaloid) derived from an oripavine- and thebaine-rich mutant of a Papaver somniferum poppy. The poppy straw thus treated was charged to the extraction vessel (13) and the chamber of extraction vessel (13) was closed. A solution of 5.0 grams of potassium hydroxide in 15.0 grams of water was added to the phase separating column (14) equipped with the static mixer. Liquid dimethyl ether (ca. 1.1 L) at 50° C. was passed through the extraction vessel (13) and the resulting extract was passed through the phase separation column (14) containing the aqueous potassium hydroxide solution (directional movement into phase separation column (14) not shown). The liquid dimethyl ether layer containing the extracted materials exited through the top of the phase separation column (14) and was passed into open collection vessel (18) through pressure reduction valve (V-9). When the extraction was complete, the aqueous phase (49.2 grams) was separated through pressure reduction valve (V-8) to collection vessel (17). The remaining dimethyl ether extract in the phase separation column (14) was drained through pressure reduction valve (V-8) and combined with the dimethyl ether extract in open collection vessel (18). The dimethyl ether was vented from the open collection vessel (18) at ambient conditions to yield a slurry in water (51.7 grams). The slurry from open collection vessel (18) was acidified with 10% acetic acid solution (final pH=4.0). The acidified solution was extracted with toluene (3×10 mL), the toluene extracts were combined, and the toluene was distilled on a rotary evaporator at 60° C./40 mbar. The residues were rinsed into a sample vial and the sample was dried overnight in a vacuum oven at 55° C./40 mbar to yield 1.1 grams of a black tarry material (Extract 1).

The pH of the aqueous layer was adjusted to pH 9-10 by the addition of 28% aqueous ammonia. The mixture was then extracted with toluene (3×10 mL), the toluene extracts were combined, and the toluene distilled on a rotary evaporator at 60° C./40 mbar. The residues were rinsed into a sample vial and the sample was dried overnight in a vacuum oven at 55° C./40 mbar to yield 0.6 grams of a brown solid (Extract 2).

The potassium hydroxide aqueous phase collected in vessel (2) was neutralized by the addition of 10% acetic acid solution to pH 9.0-9.5. The mixture was allowed to cool to ambient temperature with stirring for 1 hour and the solids were then filtered. The solids were transferred to a sample vial and the sample was dried overnight in a vacuum oven at 55° C./40 mbar to yield 0.5 grams of a crystalline brown solid (Extract 3). Extracts 1 through 3 were then analyzed using the UPLC analysis method described above. Table 9 below, shows the analytical results for the isolated solid (Wt % thebaine and Wt % oripavine).

TABLE 9
Analytical results for Example 9

| | Weight | Oripavine | Thebaine assay |
|---|---|---|---|
| Sample | (grams) | Assay (Wt. %) | (Wt. %) |
| Poppy straw | 49.8 | 1.32 (0.66 gms) | 1.11 (0.55 gms) |
| Extract 1 (tars etc) | 1.1 | ND | 2.1 (0.23 gms) |
| Extract 2 (alkaloids | 0.6 | 4.0 (0.024 gms) | 78.4 (0.47 gms) |
| from DME residues) | | | |
| Extract 3 (alkaloids | 0.5 | 90.6 (0.45 gms) | ND |
| from KOH extract) | | | |

The recovery of oripavine was 73% of theoretical. The recovery of thebaine was 90% theoretical.

### Example 10

### Two-Step Extraction of Oripavine and Thebaine From a Poppy Straw Rich in Thebaine and Oripavine

Milled, oripavine- and thebaine-rich poppy straw (50.0 grams) from an oripavine- and thebaine-rich mutant of a Papaver somniferum poppy (EVE) was charged to the extraction vessel (13) and the chamber of extraction vessel (13) was closed. Liquid dimethyl ether (ca. 1.1 L) at 25° C. was passed through the extraction vessel (13) and the resulting extract was passed through the pressure reduction valve (V-7) to open collection vessel (16). The dimethyl ether was vented from the open collection vessel (16) to yield an oil, which on further drying under vacuum yielded a tar (Extract 1; 0.8 grams).

The extracted poppy straw was removed from the extraction vessel (13) and mixed with a slurry of 1.1 grams of calcium hydroxide in 51.2 grams of water. The poppy straw thus treated was re-charged to the extraction vessel (13) and the chamber of extraction vessel (13) was closed. A solution of 5.3 grams of potassium hydroxide in 5.4 grams of water was added to the phase separating column (14). Liquid dimethyl ether (ca. 1.1 L) at 50° C. was passed through the extraction vessel (13) and the resulting extract was passed through the phase separation column (14) containing the aqueous potassium hydroxide solution (directional movement into phase separation column (14) not shown). The liquid dimethyl ether layer containing the extracted materials exited through the top of the phase separation column (14) and was passed into open collection vessel (18) through pressure reduction valve (V-9). When the extraction was complete, the first aqueous phase (38.5 grams) was separated through pressure reduction valve (V-8) to collection vessel (17). The remaining dimethyl ether extract in the phase separation column (14) was drained through pressure reduction valve (V-8) and combined with the dimethyl ether extract in open collection vessel (18). The dimethyl ether was vented from the open collection vessel (18) to yield a slurry in water weighing 41.6 grams (second aqueous phase). The water was evaporated on a rotary evaporator at 60° C./40 mbar to yield 1.2 grams of a solid (Extract 2).

The pH of the first aqueous phase from the phase separation column (14) was adjusted to pH 9-10 and an attempt to isolate the product by extraction into acetonitrile was unsuccessful. The acetonitrile was distilled from the mixture on a rotary evaporator and the mixture was allowed to cool. The solids were collected by filtration and dried in a vacuum oven at 60° C./40 mbar overnight (Extract 3). Extracts 1 through 3 were then analyzed using the UPLC analysis method described above. Table 10 below, shows the analytical results for the isolated solid (Wt % thebaine and Wt % oripavine).

TABLE 10
Analytical results for Example 10

| | Weight | Thebaine | Oripavine Assay |
|---|---|---|---|
| Sample | (grams) | Assay (Wt. %) | (Wt. %) |
| Poppy straw | 50.0 | 1.11 (0.0056 gms) | 1.32 (0.66 gms) |
| Extract 1 | 0.8 | 3.0 (0.024 gms) | 0.9 (0.0072 gms) |
| Extract 2 | 1.2 | 49.3 (0.59 gms) | 7.3 (0.088 gms) |
| Extract 3 | 0.3 | 0.3 (0.0009 gms) | 91.2 (0.27 gms) |

The recovery of thebaine was 106% of theoretical. The recovery of oripavine was 54% of theoretical.

### Example 11

### Two-Step Extraction of Oripavine and Thebaine From a Poppy Straw Rich in Thebaine and Oripavine Using a Higher Extraction Temperature (80° C.)

Milled, oripavine- and thebaine-rich poppy straw (49.9 grams) from an oripavine- and thebaine-rich mutant of a Papaver somniferum poppy was charged to the extraction vessel (13) and the chamber of extraction vessel (13) was closed. Liquid dimethyl ether (ca. 1.1 L) at 50° C. was passed through the extraction vessel (13) and the resulting extract was passed through the pressure reduction valve (V-7) to open collection vessel (16). The dimethyl ether was vented from the open collection vessel (16) at ambient conditions to yield an oil, which on further drying under vacuum yielded a tar (1.2 grams).

The extracted poppy straw was removed from the extraction vessel (13) and mixed with a slurry of 1.0 grams of calcium hydroxide in 51.0 grams of water. The poppy straw thus treated was re-charged to the extraction vessel (13) and the chamber of extraction vessel (13) was closed. Liquid dimethyl ether (ca. 1.1 L) at 80° C. was passed through the extraction vessel (13) and the resulting extract was passed through pressure reduction valve (V-7) to open collection vessel (16). The dimethyl ether was vented from the open collection vessel (16) at ambient conditions to yield 67.4 grams of a slurry in water. The pH of the aqueous solution was adjusted to pH 13.4 by the addition of 10% potassium hydroxide solution. The mixture was then extracted five times with 20 mL of toluene. The toluene extracts were combined and the toluene was removed on a rotary evaporator to yield 0.7 grams of a solid. The water from the remaining aqueous solution was removed on a rotary evaporator to yield 1.9 grams of a solid. The solid was then analyzed using the UPLC analysis method described above. Table 11 below, shows the analytical results for the isolated solid (Wt % thebaine and Wt % oripavine).

TABLE 11
Analytical results for Example 11

| | Weight | Thebaine | Oripavine assay |
|---|---|---|---|
| Sample | (grams) | Assay (Wt. %) | (Wt. %) |
| Poppy straw | 49.9 | 1.11 (0.55 gms) | 1.32 (0.66 gms) |
| Extract 1 | 1.2 | 4.6 (0.055 gms) | 1.6 (0.019 gms) |
| Toluene extract | 0.7 | 62.5 (0.44 gms) | 0.8 (0.006 gms) |
| Aqueous extract | 1.9 | 0.3 (0.006 gms) | 24.1 (0.46 gms) |

The recovery of thebaine was 80% of theoretical. The recovery of oripavine was 69% of theoretical.

### Example 12

### One-Step Extraction of Morphine From Morphine Poppy Straw; On-Line Extraction into Potassium Hydroxide Solution

A slurry of 1.18 grams of calcium hydroxide in 51.0 grams of water was prepared and mixed with 49.7 grams of a morphine-rich poppy straw from a morphine Papaver somniferum poppy. The poppy straw thus treated was charged to the extraction vessel (13) and the chamber of extraction vessel (13) was closed. A solution of 5.2 grams of potassium hydroxide in 17.1 grams of water was added to the phase separating column (14) equipped with the static mixer. Liquid dimethyl ether (ca. 1.1 L) at 50° C. was passed through the extraction vessel (13) and the resulting extract was passed through the phase separation column (14) containing the aqueous potassium hydroxide solution (directional movement into phase separation column (14) not shown). The liquid dimethyl ether layer containing the extracted materials exited through the top of the phase separation column (14) and was passed into open collection vessel (18) through pressure reduction valve (V-9). When the extraction was complete, the aqueous phase (47.2 grams) was separated through pressure reduction valve (V-8) to collection vessel (17). The remaining dimethyl ether extract in the phase separation column was drained through pressure reduction valve (V-8) and combined with the dimethyl ether extract in open collection vessel (18). The dimethyl ether gas vented at ambient conditions from the open collection vessel (18) to yield a slurry in water (41.3 grams). The slurry from open collection vessel (18) was acidified with 10% acetic acid solution (final pH=4.0). The acidified solution was extracted with toluene (3×10 mL), the toluene extracts were combined, and the toluene was distilled from the extracts on a rotary evaporator at 60° C./40 mbar. The residues were rinsed into a sample vial and the sample was dried overnight in a vacuum oven at 55° C./40 mbar to yield 0.7 grams of a black tarry material (Extract 1).

The pH of the aqueous layer was adjusted to pH 9-10 by the addition of 28% aqueous ammonia. The mixture was then extracted with toluene (3×10 mL), the toluene extracts were combined, and the toluene distilled from the extracts on a rotary evaporator at 60° C./40 mbar. The residues were rinsed into a sample vial and the sample was dried overnight in a vacuum oven at 55° C./40 mbar to yield 0.7 grams of a brown solid (Extract 2).

The potassium hydroxide aqueous phase collected in vessel (2) was neutralized by the addition of 10% acetic acid solution to pH 9.0. The mixture was allowed to cool to ambient temperature with stirring for 1 hour and the solids were then filtered. The solids were transferred to a sample vial and the sample was dried overnight in a vacuum oven at 55° C./40 mbar to yield 0.6 grams of a crystalline brown solid (Extract 3). Extracts 1 through 3 were then analyzed using the UPLC analysis method described above. Table 12 below, shows the analytical results for the isolated solid (Wt % thebaine, WT % codeine and Wt % morphine).

TABLE 12
Analytical results for Example 12

| | Weight | Morphine | Thebaine | Codeine |
|---|---|---|---|---|
| Sample | (grams) | Assay (Wt. %) | Assay (Wt. %) | Assay (Wt. %) |
| Poppy straw | 49.7 | 1.74 (0.87 gms) | | |
| Extract 1 | 0.7 | ND | 0.3 (0.002 gms) | |
| Extract 2 (DME | 0.7 | 2.7 (0.19 gms) | 5.0 (0.035 gms) | 3.6 (0.0025 gms) |
| extract, alkaloids) | | | | |
| Extract 3 (KOH/ | 0.5 | 83.0 (0.42 gms) | 0.4 (0.002 gms) | |
| Aqueous extr.) | | | | |

The recovery of morphine was 59% of theoretical.

### Example 13

### Two-Step Extraction of Morphine From Morphine Poppy Straw

Milled, morphine-rich poppy straw (50.0 grams) from a morphine Papaver somniferum poppy was charged to the extraction vessel (13) and the chamber of extraction vessel (13) was closed. Liquid dimethyl ether (ca. 1.1 L) at 25° C. was passed through the extraction vessel (13) and the resulting extract was passed through the pressure reduction valve (V-7) to open collection vessel (16). The dimethyl ether was vented from the open collection vessel (16) to yield an oil, which on further drying under vacuum yielded a tar (Extract 1; 0.9 grams).

The extracted poppy straw was removed from the extraction vessel (13) and mixed with a slurry of 1.1 grams of calcium hydroxide in 52.2 grams of water. The poppy straw thus treated was re-charged to the extraction vessel (13) and the chamber of extraction vessel (13) was closed. A solution of 5.3 grams of potassium hydroxide in 5.9 grams of water was added to the phase separating column (14). Liquid dimethyl ether (ca. 1.1 L) at 50° C. was passed through the extraction vessel (13) and the resulting extract was passed through the phase separation column (14) containing the aqueous potassium hydroxide solution (directional movement into phase separation column (14) not shown). The liquid dimethyl ether layer containing the extracted materials exited through the top of the phase separation column (14) and was passed into open collection vessel (18) through pressure reduction valve (V-9). When the extraction is complete, the aqueous phase (38.4 grams, Aqueous Phase 1) was separated through pressure reduction valve (V-8) to collection vessel (17). The pH of the aqueous phase from the phase separation column (14) was adjusted to pH 9-10. The resulting solids were collected by filtration and dried in a vacuum oven at 60° C./40 mbar overnight (Extract 2; 0.5 grams).

The remaining dimethyl ether extract in the phase separation column (14) was drained through pressure reduction valve (V-8) and combined with the dimethyl ether extract in open collection vessel (18). The dimethyl ether was vented from the open collection vessel to yield a slurry in water weighing 36.1 grams (Aqueous Phase 2). The water was evaporated on a rotary evaporator at 60° C./40 mbar to yield 0.8 grams of a solid Extract 3. Extracts 1 through 3 were then analyzed using the UPLC analysis method described above. Table 13 below, shows the analytical results for the isolated solid (Wt % thebaine, Wt % codeine and Wt % morphine).

TABLE 13
Analytical results for Example 13.

| | Weight | Morphine | Thebaine | Codeine |
|---|---|---|---|---|
| Sample | (grams) | Assay (Wt. %) | Assay (Wt. %) | Assay (Wt. %) |
| Poppy straw | 50.0 | 1.74 (0.87 gms) | | |
| Extract 1 | 0.9 | 0.3 (0.003 gms) | 0.4 (0.004 gms) | |
| Extract 2 | 0.5 | 81.1 (0.41 gms) | 0.3 (0.002 gms) | |
| Extract 3 | 0.8 | 13.3 (0.11 gms) | 7.3 (0.58 gms) | 3.7 (0.30 gms) |

The recovery of morphine was 59% of theoretical.

### Example 14

### One-Step Extraction of Thebaine From a Thebaine-Rich Poppy Straw On-Line Extraction into Organic Acid Solution

In a suitable container, a slurry of 19.4 grams of calcium hydroxide in 320 grams of water was prepared and mixed with 320.5 grams of a dried, milled, thebaine-rich poppy straw from a thebaine-rich mutant of a Papaver somniferum poppy to treat the poppy straw. The poppy straw thus treated was charged to an extraction vessel and the chamber of the extraction vessel was closed. A solution of 20% w/v potassium hydroxide (400 ml) was added into a first phase separation column. A solution of citric acid (500 ml, 20% w/v) was added into a second phase separation column, where each, first and second phase separation columns were equipped with a static mixer. Liquid dimethyl ether (ca. 5.5 L) at approximately 50° C. was passed through the extraction vessel and the resulting extract was passed through the phase separation columns in series, first through the first phase separation column containing aqueous potassium hydroxide, then through the second phase separation column containing the aqueous citric acid solution. The spent liquid dimethyl ether layer containing the non-alkaloid tarry materials exited through the top of the phase separation columns and was passed into an open collection vessel through pressure reduction valves. When the extraction was complete, the aqueous phase from both columns was drained to the collection vessels. The volumes of potassium hydroxide and citric acid solutions were 474 ml and 262 ml, respectively. Thebaine in the citric acid solution was precipitated in the presence of ethanol (approximately 10% of the volume of the batch) using 40% w/v potassium hydroxide solution. Solid isolated was rinsed with warm water (55° C., ˜10 L/kg) and dried at 50° C. under vacuum overnight.

The concentrate of poppy straw (CPS) solid was assayed at 97.9% against a pure standard. The primary extraction yield, based on the UPLC analysis of the extract streams, was approximately 93%. The repeat of this run without the potassium hydroxide phase separation column gave a CPS solid with an assay of 94.3%. The primary extraction yield for this repeated run was 98%, based on the UPLC analysis of the extract streams.

The spent poppy straw was collected for direct use as combustible fuel.

### Example 15

### Prophetic Example

### Extraction of Thebaine From Roots of Papaver Bracteatum

A slurry of 1.15 grams of calcium hydroxide in 50.3 grams of water is prepared and mixed with 49.1 grams of milled or crushed roots from a Papaver bracteatum poppy. The poppy root thus treated is charged to the extraction vessel (13) and the chamber of extraction vessel (13) is closed. A solution of 5.4 grams of phosphoric acid (85%) in 15.6 grams of water is added to the phase separating column (14) equipped with the static mixer. Liquid dimethyl ether (ca. 1.1 L) at 50° C. is passed through the extraction vessel (13) and the resulting extract is passed through the phase separation column (14) containing the aqueous phosphoric acid solution. The liquid dimethyl ether layer containing the extracted materials exits through the top of the phase separation column (14) and is passed into open collection vessel (18) through pressure reduction valve (V-9). When the extraction is complete, the aqueous phase is separated through pressure reduction valve (V-8) to collection vessel (17). The remaining dimethyl ether extract in the phase separation column (14) is drained through pressure reduction valve (V-8) and combined with the dimethyl ether extract in open collection vessel (18). The dimethyl ether is vented from the open collection vessel to yield a slurry in water. The water is evaporated on a rotary evaporator at 60° C./40 mbar to yield a residue.

The pH of the phosphoric acid extract is adjusted to a pH in the range of pH 9-10 by the addition of 28% aqueous ammonia. The resulting thebaine is isolated by filtration or by extraction into toluene (3×10 mL), combining the toluene extracts, and distilling the toluene from the extracts on a rotary evaporator at 60° C./40 mbar. To obtain assay (wt %) data, the solid is analyzed using the UPLC analysis method described above. The spent poppy root is collected for direct use as combustible fuel.

### Example 16

### Prophetic Example

### Extraction of Noscapine and Morphine From Noscapine-Morphine Rich Poppy

A slurry of 2% by weight relative to the weight of poppy straw of calcium hydroxide in 100% water is prepared and mixed with a poppy straw containing both noscapine (non-phenolic alkaloid) and morphine (phenolic alkaloid) derived from a morphine- and noscapine-rich mutant of a Papaver somniferum poppy. The poppy straw thus treated is charged to the extraction vessel (13) and the chamber of extraction vessel (13) is closed. A solution of 30% aqueous potassium hydroxide is added to the phase separating column (14) equipped with the static mixer. Liquid dimethyl ether at 50° C. is passed through the extraction vessel (13) and the resulting extract is passed through the phase separation column (14) containing the aqueous potassium hydroxide solution. The liquid dimethyl ether layer containing the total extract exits through the top of the phase separation column (14) and is passed into open collection vessel (18) through pressure reduction valve (V-9). When the extraction is complete, the aqueous phase is separated through pressure reduction valve (V-8) to collection vessel (17). The remaining dimethyl ether extract in the phase separation column (14) is drained through pressure reduction valve (V-8) and combined with the dimethyl ether extract in open collection vessel (18). The dimethyl ether gas is vented from the open collection vessel (18) at ambient conditions to yield a slurry in water. The slurry from collection vessel (18) is acidified with 10% acetic acid solution. The acidified solution is extracted with toluene, the toluene extracts are combined, and the toluene is distilled from the extracts on a rotary evaporator at 60° C./40 mbar. The residues are rinsed into a sample vial and the sample is dried overnight in a vacuum oven at 55° C./40 mbar to yield Extract 1.

The pH of the aqueous layer is adjusted to pH 9.0-10.0 by the addition of 28% aqueous ammonia. The mixture is then extracted with toluene (3×10 mL), the toluene extracts are combined, and the toluene distilled from the extracts on a rotary evaporator at 60° C./40 mbar. The residues are rinsed into a sample vial and the sample is dried in a vacuum oven at 55° C./40 mbar to yield noscapine as a residue.

The potassium hydroxide aqueous phase (collected in vessel 17) is neutralized by the addition of 10% acetic acid solution to pH 9.0-9.5. The mixture is allowed to cool to ambient temperature with stirring for 1 hour and solids are then filtered. The solids are transferred to a sample vial and the sample is dried overnight in a vacuum oven at 55° C./40 mbar to yield morphine as a residue. To obtain assay (wt %) data for the extracted alkaloids, the solid is analyzed using the UPLC analysis method described above.

The spent poppy straw is collected for direct use as combustible fuel.

### Example 17

### Two-Step Extraction (i.e., Including Base Pre-Treatment Step) of Codeine From a Codeine-Rich Poppy Straw

In a suitable container, 27.0 grams of potassium hydroxide was dissolved in ethanol to a final volume of 300 mL and mixed with 300.2 grams of a dried, milled, codeine-rich poppy straw from a codeine-rich mutant of a Papaver somniferum poppy to treat the poppy straw. The poppy straw thus treated was immediately charged to an extraction vessel and the chamber of the extraction vessel was closed. Liquid dimethyl ether (ca. 5.5 L) at approximately 50° C. was passed through the extraction vessel and the resulting extract was passed through pressure reduction valves into an open collection vessel. Following evaporation of the dimethyl ether the remaining extract was rotary evaporated to dryness and further dried overnight in a vacuum oven at 45° C. and a pressure of −100 mbar to yield a brown glassy solid, (20.8 grams).

The crude extract was homogenized and assayed at 48.0% (as is) codeine. The primary extraction yield, based on UPLC analysis of the extract, was approximately 95%.

### Example 18

### One-Step Extraction (i.e., Excluding Base Pretreatment Step) of Noscapine From a Noscapine-Rich Poppy Straw

Dried, milled, noscapine-rich poppy straw (290.4 grams) from a noscapine-rich mutant of a Papaver somniferum poppy was charged to an extraction vessel without pre-treatment and the chamber of the extraction vessel was closed. After standing in the extraction vessel for 1 hour at approximately 50° C., liquid dimethyl ether (ca. 11 L) at approximately 50° C. was passed through the extraction vessel and the resulting extract was passed through pressure reduction valves into an open collection vessel. Following evaporation of the dimethyl ether the remaining extract was rotary evaporated to dryness and further dried overnight in a vacuum oven at 45° C. and a pressure of −100 mbar to yield a brown waxy solid, (10.5 grams). The primary extraction yield, based on initial and post extraction analysis of the straw noscapine content, was approximately 82%.

The solid was dissolved in toluene (200 mL) and after multiple liquid/liquid partitioning steps, noscapine was precipitated from a phosphoric acid extract by addition of 40% w/v potassium hydroxide solution. Solid isolated was rinsed with water (5 mL) and dried overnight in a vacuum oven set at a temperature of 45° C. and a pressure of −100 mbar to yield a crude noscapine solid (3.2 grams) assayed at 73.6% noscapine.

### Example 19

### One-Step Extraction of Codeine From a Codeine-Rich Poppy Straw and Collection of Extracted Alkaloids Using pH 6.0 Phosphate Buffer

In a suitable container, a slurry of 18.0 grams of calcium hydroxide in 300 grams of water was prepared and mixed with 300.3 grams of a dried, milled, codeine-rich poppy straw from a codeine-rich mutant of a Papaver somniferum poppy to treat the poppy straw. The poppy straw thus treated was charged to an extraction vessel and the chamber of the extraction vessel was closed. 2M phosphate buffer solution (pH 6.0, 300 ml) was added to both phase separation columns, which were equipped with static mixers. Liquid dimethyl ether (ca. 5.5 L) at approximately 50° C. was passed through the extraction vessel and the resulting extract was passed through the phase separation columns in series, first through the first phase separation column, then through the second phase separation column. The resultant liquid dimethyl ether extract exited through the top of the phase separation columns and was passed into an open collection vessel through pressure reduction valves. When the extraction was complete, the aqueous phase from both columns was drained to the collection vessels. The solution volumes recovered from phase separation column 1 and phase separation column 2 were 312 mL and 305 mL, respectively and, after evaporation of residual dimethyl ether, the volume of extract in acetic acid was 187 mL. UPLC assay indicated that 87% of codeine and 38% of thebaine initially present in the straw was collected in the columns.

The contents of the two columns were combined (595 mL), heated to 55° C. and extracted with toluene (180 mL) at pH 10.5 (adjusted by addition of 40% w/v aqueous potassium hydroxide). The phases were split and the aqueous phase was extracted a second time with toluene (60 mL). The toluene extracts were combined, rotary evaporated to dryness and dried overnight in vacuo at 45° C. and −95 kPa to yield codeine (9.72 g, codeine content 90.32% on dry basis, thebaine content 4.81% w/w on dry basis). The yield of codeine was 82.3% from straw, whereas the yield of thebaine was 34.5% from straw.

One of ordinary skill in the art will appreciate further features and advantages of the invention based on the above-described embodiments. Accordingly, the invention is not to be limited by what has been particularly shown and described as examples. While the foregoing specification teaches the principles of the present invention, it will be understood that the practice of the invention encompasses all of the usual variations, adaptations and/or modifications as come within the scope of the following claims and their equivalents.

What is claimed is:

1. A process for the extraction of one or more of an alkaloid or a non-alkaloid material from a plant of the Papaver species comprising the steps of:
(a) providing a plant of the Papaver species comprising one or more alkaloid(s), one or more non-alkaloid material or mixtures thereof; and
(b) contacting the plant of the Papaver species with liquefied, sub-critical or super-critical dimethyl ether; to yield an extract comprising the liquefied, sub-critical or super-critical dimethyl ether and the extracted alkaloid or non-alkaloid materials.

2. A process as in claim 1, wherein the plant of the Papaver species is selected from the group consisting of (a) poppy straw resulting from the end of the growth cycle of the Papaver somniferum poppy, or mutants thereof, (b) poppy straw resulting from the end of the growth cycle of a thebaine-rich mutant of the Papaver somniferum poppy, (c) poppy straw resulting from the end of the growth cycle of a codeine-rich mutant of the Papaver somniferum poppy, (d) poppy straw resulting from the end of the growth cycle of an oripavine- and thebaine-rich mutant of the Papaver somniferum poppy, (e) poppy straw resulting from the end of the growth cycle of morphine-rich Papaver somniferum poppy, (f) poppy straw resulting from the end of the growth cycle of a noscapine-rich mutant of the Papaver somniferum poppy, (g) the roots of the Papaver bracteatum poppy, (h) opium or extracts thereof, and (i) tissue cultures of plant material enriched in secondary metabolites.

3. A process as in claim 1, wherein the plant of the Papaver species is selected from the group consisting of the poppy straw of the top 10 centimeters of the Papaver somniferum poppy or mutants thereof, and the roots of the Papaver bracteatum poppy.

4. A process as in claim 1, wherein the plant of the Papaver species is selected from the group consisting of poppy straw resulting from the end of the growth cycle of a thebaine-rich mutant of a Papaver somniferum poppy, poppy straw resulting from the end of the growth cycle of a codeine-rich mutant of a Papaver somniferum poppy, poppy straw resulting from the end of the growth cycle of an oripavine- and thebaine-rich mutant of a Papaver somniferum poppy, poppy straw resulting from the end of the growth cycle of a morphine-rich Papaver somniferum poppy and poppy straw from the end of the growth cycle of a noscapine-rich Papaver somniferum poppy.

5. A process as in claim 1, further comprising the step of contacting the plant of the Papaver species with an organic or inorganic base, prior to the step of contacting the plant with the liquefied, sub-critical or super-critical dimethyl ether.

6. A process as in claim 5, where the organic or inorganic base is selected from the group consisting of triethylamine, calcium hydroxide, ammonia, potassium hydroxide, sodium hydroxide, potassium carbonate, sodium carbonate and sodium bicarbonate.

7. A process as in claim 5, wherein the organic or inorganic base is aqueous calcium hydroxide.

8. A process as in claim 1, further comprising the step of isolating the one or more extracted alkaloids.

9. A process as in claim 8, wherein the one or more extracted alkaloids are isolated by filtration; or by evaporation of the liquefied, sub-critical or supercritical dimethyl ether; or by extraction with an organic solvent; or by extraction with an aqueous base; or by extraction with an aqueous base followed by pH adjustment of the resulting mixture and filtration; or by extraction with an aqueous acid followed by pH adjustment of the resulting mixture and filtration; or by evaporation of the liquefied, sub-critical or supercritical dimethyl ether followed by reaction of the one or more alkaloids with an acid to yield the corresponding acid addition salt of the one or more alkaloids, followed optionally by filtration.

10. A process as in claim 8, wherein the process steps are run under continuous process conditions.

11. A process for the extraction of one or more alkaloids comprising the steps of
(a) providing a plant of the Papaver species comprising alkaloids;
(b) contacting the plant of the Papaver species with an organic or inorganic base; to yield a base-treated poppy plant; and
(c) contacting the base-treated poppy plant with liquefied, sub-critical or super-critical dimethyl ether; to yield an extract comprising the liquefied, sub-critical or super-critical dimethyl ether, and one or more extracted alkaloids.

12. A process as in claim 11, wherein the plant of the Papaver species is selected from the group consisting of (a) poppy straw resulting from the end of the growth cycle of the Papaver somniferum poppy, or mutants thereof, (b) poppy straw resulting from the end of the growth cycle of a thebaine-rich mutant of the Papaver somniferum poppy, (c) poppy straw resulting from the end of the growth cycle of a codeine-rich mutant of the Papaver somniferum poppy, (d) poppy straw resulting from the end of the growth cycle of an oripavine- and thebaine-rich mutant of the Papaver somniferum poppy, (e) poppy straw resulting from the end of the growth cycle of a noscapine-rich mutant of the Papaver somniferum poppy, (f) the roots of the Papaver bracteatum poppy, (g) opium or extracts thereof, and (h) tissue cultures of plant material enriched in secondary metabolites.

13. A process as in claim 11, wherein the plant of the Papaver species is selected from the group consisting of the poppy straw of the top 10 centimeters of the Papaver somniferum poppy or mutants thereof, and the roots of the Papaver bracteatum poppy.

14. A process as in claim 11, wherein the plant of the Papaver species is selected from the group consisting of poppy straw resulting from the end of the growth cycle of a thebaine-rich mutant of a Papaver somniferum poppy, poppy straw resulting from the end of the growth cycle of a codeine-rich mutant of a Papaver somniferum poppy, poppy straw resulting from the end of the growth cycle of an oripavine- and thebaine-rich mutant of a Papaver somniferum poppy, poppy straw resulting from the end of the growth cycle of a morphine-rich mutant of a Papaver somniferum poppy and poppy straw resulting from the end of the growth cycle of a noscapine-rich mutant of a Papaver somniferum poppy.

15. A process as in claim 11, wherein the organic or inorganic base is selected from the group consisting of triethylamine, calcium hydroxide, ammonia, potassium hydroxide, sodium hydroxide, potassium carbonate, sodium carbonate and sodium bicarbonate.

16. A process as in claim 11, wherein the organic or inorganic base is aqueous calcium hydroxide.

17. A process as in claim 11, further comprising the step of isolating the one or more extracted alkaloids.

18. A process as in claim 17, wherein the one or more extracted alkaloids are isolated by filtration or by evaporation of the liquefied, sub-critical or super-critical dimethyl ether or by evaporation of the liquefied, sub-critical or super-critical dimethyl ether followed by reaction of the one or more alkaloids with an acid to yield the corresponding acid addition salt of the one or more alkaloids, followed optionally by filtration.

19. A process as in claim 17, wherein the one or more extracted alkaloids are isolated by extraction with an organic solvent or an aqueous base; followed by filtration or evaporation of the organic solvent; to yield the one or more alkaloids.

20. A process as in claim 17, wherein the one or more extracted alkaloids are isolated by extraction with an aqueous base followed by pH adjustment of the resulting mixture and filtration; or by extraction with an aqueous acid followed by pH adjustment of the resulting mixture and filtration.

21. A process as in claim 17, further comprising the additional steps of
(a) contacting the extract with an aqueous base or buffer; to yield a first biphasic mixture comprising a first aqueous phase and a first organic phase; wherein the first biphasic mixture is substantially free of emulsion; wherein the first aqueous phase of the first biphasic mixture is enriched in phenolic alkaloids; and wherein the first organic phase of the first biphasic mixture is enriched in non-phenolic alkaloids;
(b) separating the first aqueous phase and first organic phase one from the other;
(c) isolating the phenolic alkaloids from the first aqueous phase by adjusting the pH of the first aqueous phase to a pH in the range of from about pH 7.5 to about pH 11.5; and filtering the resulting mixture;
(d) contacting the first organic phase of the first biphasic mixture with an aqueous acid; to yield a second biphasic mixture comprising a second aqueous phase and a second organic phase; wherein the second biphasic mixture is substantially free of emulsion; and wherein the second aqueous phase of the second biphasic mixture is enriched in non-phenolic alkaloids;
(e) separating the second aqueous phase and second organic phase one from the other; and
(e) isolating the non-phenolic alkaloids from the second aqueous phase by adjusting the pH of the second aqueous phase to a pH in the range of from about pH 7.5 to about pH 11.5; and filtering the resulting mixture.

22. A process as in claim 17, wherein the one or more extracted alkaloids are isolated using a continuous process.

23. A process as in claim 17, wherein the one or more extracted alkaloids are isolated by countercurrent extraction, under continuous process conditions.

24. A process as in claim 21, wherein the aqueous acid is an aqueous inorganic acid, an aqueous organic acid or a mixture thereof.

25. A process as in claim 24, wherein the aqueous acid is an aqueous inorganic acid.

26. A process as in claim 25, wherein the aqueous inorganic acid is phosphoric acid, hydrochloric acid, sulfuric acid, or a mixture thereof.

27. A process as in claim 26, wherein the aqueous inorganic acid is phosphoric acid.

28. A process as in claim 24, wherein the aqueous acid is an aqueous organic acid.

29. A process as in claim 28, wherein the aqueous organic acid is lactic acid, formic acid, acetic acid, citric acid, oxalic acid, uric acid or mixtures thereof.

30. A process as in claim 29, wherein the aqueous organic acid is citric acid, oxalic acid or mixtures thereof.

31. A process for the extraction of one or more alkaloids comprising the steps of
(a) providing a plant of the Papaver species comprising one or more alkaloids, one or more non-alkaloid materials or mixtures thereof;
(b) contacting the plant of the Papaver species with a first liquefied, sub-critical or super-critical fluid; to yield i) a non-alkaloid material depleted poppy plant and ii) a first extract comprising the first liquefied, sub-critical or super-critical fluid and one or more extracted non-alkaloid materials;
(c) contacting the non-alkaloid material depleted poppy plant with an organic or inorganic base; to yield a base-treated, non-alkaloid material depleted poppy plant;
(d) contacting the base-treated, non-alkaloid material depleted poppy plant with a second liquefied, sub-critical or super-critical fluid ; to yield a second extract comprising the second liquefied, sub-critical or super-critical, water, and one or more extracted alkaloids, wherein at least one of the first and/or second liquefied, sub-critical or super-critical fluids is liquefied, sub-critical or super-critical dimethyl ether.

32. A process as in claim 31, wherein the plant of the Papaver species is selected from the group consisting of (a) poppy straw resulting from the end of the growth cycle of the Papaver somniferum poppy, or mutants thereof, (b) poppy straw resulting from the end of the growth cycle of a thebaine-rich mutant of the Papaver somniferum poppy, (c) poppy straw resulting from the end of the growth cycle of a codeine-rich mutant of the Papaver somniferum poppy, (d) poppy straw resulting from the end of the growth cycle of an oripavine- and thebaine-rich mutant of the Papaver somniferum poppy, (e) the roots of the Papaver bracteatum poppy, (f) poppy straw resulting from the end of the growth cycle of a noscapine-rich mutant of the Papaver somniferum poppy, (g) opium or extracts thereof, and (h) tissue cultures of plant material enriched in secondary metabolites.

33. A process as in claim 31, wherein the plant of the Papaver species is selected from the group consisting of the poppy straw of the top 10 centimeters of the Papaver somniferum poppy or mutants thereof, and the roots of the Papaver bracteatum poppy.

34. A process as in claim 31, wherein the plant of the Papaver species is selected from the group consisting of poppy straw resulting from the end of the growth cycle of a thebaine-rich mutant of a Papaver somniferum poppy, poppy straw resulting from the end of the growth cycle of a codeine-rich mutant of a Papaver somniferum poppy, poppy straw resulting from the end of the growth cycle of an oripavine- and thebaine-rich mutant of a Papaver somniferum poppy, poppy straw resulting from the end of the growth cycle of a morphine-rich mutant of a Papaver somniferum poppy and poppy straw resulting from the end of the growth cycle of a noscapine-rich mutant of a Papaver somniferum poppy.

35. A process as in claim 31, wherein the organic or inorganic base is selected from the group consisting of triethylamine, calcium hydroxide, ammonia, potassium hydroxide, sodium hydroxide, potassium carbonate, sodium carbonate and sodium bicarbonate.

36. A process as in claim 31, wherein the organic or inorganic base is aqueous calcium hydroxide.

37. A process as in claim 31, further comprising the additional step of isolating the one or more extracted alkaloids.

38. A process as in claim 37, wherein the one or more extracted alkaloids are isolated by filtration or by evaporation of the liquefied, sub-critical or super-critical dimethyl ether or by evaporation of the liquefied, sub-critical or super-critical dimethyl ether followed by reaction of the one or more alkaloids with an acid to yield the corresponding acid addition salt of the one or more alkaloids, followed optionally by filtration.

39. A process as in claim 37, wherein the one or more extracted alkaloids are isolated by extraction with an organic solvent or an aqueous base; followed by filtration or evaporation of the organic solvent; to yield the one or more alkaloids.

40. A process as in claim 37, wherein the one or more extracted alkaloids are isolated by extraction with an aqueous base followed by pH adjustment of the resulting mixture and filtration; or by extraction with an aqueous acid followed by pH adjustment of the resulting mixture and filtration.

41. A process as in claim 37, further comprising the steps of
(a) contacting the extract comprising the liquefied, sub-critical or super-critical dimethyl ether, and the one or more extracted alkaloids with an aqueous base or buffer; to yield a first biphasic mixture comprising a first aqueous phase and a first organic phase; wherein the first biphasic mixture is substantially free of emulsion; wherein the first aqueous phase of the first biphasic mixture is enriched in phenolic alkaloids; and wherein the first organic phase of the first biphasic mixture is enriched in non-phenolic alkaloids;
(b) separating the first aqueous phase and first organic phase one from the other;
(c) isolating the phenolic alkaloids from the first aqueous phase by adjusting the pH of the first aqueous phase to a pH in the range of from about pH 7.5 to about pH 11.5; and filtering the resulting mixture;
(d) contacting the first organic phase of the first biphasic mixture with an aqueous inorganic or organic acid; to yield a second biphasic mixture comprising a second aqueous phase and a second organic phase; wherein the second biphasic mixture is substantially free of emulsion; and wherein the second aqueous phase of the second biphasic mixture is enriched in non-phenolic alkaloids;
(e) separating the second aqueous phase and second organic phase one from the other;
(e) isolating the non-phenolic alkaloids from the second aqueous phase by adjusting the pH of the second aqueous phase to a pH in the range of from about 7.5 to about pH 11.5; and filtering the resulting mixture.

42. A process as in claim 37, wherein the one or more extracted alkaloids are isolated using a continuous process.

43. A process as in claim 37, wherein the one or more extracted alkaloids are isolated by countercurrent extraction, under continuous process conditions.

44. A process as in claim 41, wherein the aqueous acid is an aqueous inorganic acid, an aqueous organic acid or a mixture thereof.

45. A process as in claim 44, wherein the aqueous acid is an aqueous inorganic acid.

46. A process as in claim 45, wherein the aqueous inorganic acid is phosphoric acid, hydrochloric acid, sulfuric acid, or a mixture thereof.

47. A process as in claim 46, wherein the aqueous inorganic acid is phosphoric acid.

48. A process as in claim 44, wherein the aqueous acid is an aqueous organic acid.

49. A process as in claim 48, wherein the aqueous organic acid is lactic acid, formic acid, acetic acid, citric acid, oxalic acid, uric acid or mixtures thereof.

50. A process as in claim 49, wherein the aqueous organic acid is citric acid, oxalic acid or mixtures thereof.

51. Any process for the extraction of one or more of an alkaloid or non-alkaloid material, as herein described.

52. Any process for the isolation of one or more alkaloid, as herein described.

53. A process for the extraction of one or more alkaloids, comprising the steps of
a. charging an extraction vessel with poppy plant containing non-alkaloids and alkaloids;
b. contacting the poppy plant with a first liquefied gas, sub-critical fluid or super-critical fluid to yield an alkaloid containing non-alkaloid material depleted poppy plant and an extract comprising i) the first liquefied gas, sub-critical fluid or super-critical fluid, ii) water, and iii) extracted non-alkaloid materials;
c. treating the extract of step b. with an organic base or inorganic base to yield an aqueous mixture containing base treated alkaloid containing, non-alkaloid material depleted poppy plant; and
d. contacting the aqueous mixture of step c. with a second liquefied gas, sub-critical fluid or super-critical fluid, provided that at least one of, optionally both, the first and/or second liquefied gases, sub-critical fluids or super-critical fluids is a liquefied, super-critical, or sub-critical dimethyl ether, to yield a total extract comprising i) the second liquefied gas, sub-critical fluid or super-critical fluid, ii) water and ii) extracted alkaloid(s) dissolved in a mixture of second liquefied gas, sub-critical fluid or super-critical fluid and water.

54. The process of claim 53 wherein, when present, the first or second liquefied gas, sub-critical fluid or super-critical fluid is selected from toluene, carbon dioxide, nitrous oxide or mixtures thereof.
